(19) United States
(12) Patent Application Publication
(10) Pub. No.: US 2025/0154229 A1
(43) Pub. Date: May 15, 2025

(54) MATERIALS AND METHODS FOR ENHANCED BIOPRODUCTION PROCESSES

(71) Applicant: Janssen Biotech, Inc., Horsham, PA (US)

(72) Inventors: James Vincent Price, Ardmore, PA (US); Stefanie Berges-Vorsanger, Dresher, PA (US); Kevin Clark, Moorestown, PA (US)

(21) Appl. No.: 18/834,869

(22) PCT Filed: Jan. 31, 2023
(86) PCT No.: PCT/US2023/061674

§ 371 Date: Jul. 31, 2024

Related U.S. Application Data
(60) Provisional application No. 63/352,969, filed on Jun. 16, 2022.
(60) Provisional application No. 63/305,226, filed on Jan. 31, 2022.

(51) Int. Cl. C07K 16/00; G06F 30/20

(52) U.S. Cl. CPC C07K 16/00; G06F 30/20; C07K 2317/14

(57) ABSTRACT

Provided herein is a method for reducing a lactate spike and increasing titer in a fed-batch process for producing a protein of interest, comprising reducing pyruvate (e.g., sodium pyruvate) concentration in one or more feeds to cells in a bioreactor in the fed-batch process.

### 1. CROSS-REFERENCE TO RELATED APPLICATIONS

This application claims the benefit of U.S. Provisional Patent Application No. 63/352,969, filed Jun. 16, 2022, and U.S. Provisional Patent Application No. 63/305,226, filed Jan. 31, 2022, the disclosure of each of which is incorporated by reference herein in its entirety.

### 2. SUMMARY

In one aspect, provided herein is a method for reducing a lactate spike in a fed-batch process for producing a protein of interest(s), wherein the method comprises reducing pyruvate concentration in one or more feeds to cells comprising a nucleic acid encoding the protein in a bioreactor in the fed-batch process. In another aspect, provided herein is a method for increasing the titer of a protein(s) of interest produced by cells in a fed-batch culturing process, the method comprises reducing pyruvate concentration in one or more feeds to the cells in a bioreactor in the fed-batch process. In some embodiments, the method further comprises increasing the concentration of one or more amino acids in the one or more feeds. In some embodiments, the one or more amino acids are selected from the group consisting of glutamate, valine, leucine, threonine, aspartate, and isoleucine, or a combination thereof. In some embodiments, the one or more amino acids are selected from the group consisting of glutamate, valine, and leucine, or a combination thereof. In some embodiments, the one or more amino acids are glutamate, valine, or a combination thereof. In some embodiments, the amino acid concentration is increased by about 0% to about 100%. In some embodiments, the pyruvate concentration is reduced by about 65% to about 100%. In some embodiments, the cells are CHO cells. In specific embodiments, the protein of interest is a recombinant protein. In some embodiments, the protein of interest is an antibody, a cytokine, an antigen, an enzyme, or a coagulant. In some embodiments, the protein of interest is an antibody, a cytokine, or an antigen. In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the cytokine is IL-12, IL-23, IL-1β, IL-6, IL-15, IL-2, IL-5, TNF-alpha, IL-9, or IL-17.

In another aspect, provided herein is a method of producing a protein of interest, the method comprising: (a) fed-batch culturing cells comprising a nucleic acid encoding the protein in a bioreactor under conditions sufficient for the cells to produce the protein, wherein the fed-batch culturing comprises adding a volume of one or more feeds (e.g., one or more complex feeds) comprising a pyruvate concentration that is 65% to 100% lower than the pyruvate concentration used in fed-batch culturing of the cells under the same conditions in which a lactate spike is observed; and (b) purifying the protein from the cells or liquid culture medium. In some embodiments, the concentration of one or more amino acids in the one or more feeds is increased by about 0% to about 100% relative to the fed-batch culturing of the cells under the same conditions in which a lactate spike is observed. In some embodiments, the one or more amino acids are selected from the group consisting of glutamate, valine, leucine, threonine, aspartate, and isoleucine, or a combination thereof. In some embodiments, the one or more amino acids are selected from the group consisting of glutamate, valine, and leucine, or a combination thereof. In some embodiments, the one or more amino acids are glutamate, valine, or a combination thereof. In some embodiments, the cells are CHO cells. In specific embodiments, the protein of interest is a recombinant protein. In some embodiments, the protein of interest is an antibody, a cytokine, an antigen, an enzyme, or a coagulant. In some embodiments, the protein of interest is an antibody, a cytokine, or an antigen. In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the cytokine is IL-12, IL-23, IL-1β, IL-6, IL-15, IL-2, IL-5, TNF-alpha, IL-9, or IL-17.

In another aspect, provided herein is a method for providing a digital computer simulation of a fed-batch process for producing a protein of interest, the method being implemented by one or more computing devices and comprising: receiving data characterizing the fed-batch process; initializing a model of a mammalian cell line; simulating, using the initialized model and the received data, the fed-batch process to characterize a lactate spike; identifying, based on the simulating, at least one factor contributing to the lactate spike; and providing data characterizing the identified at least one factor. In some embodiments, the provided data characterizes one or more of product composition, initial conditions, and nutrient additions of the fed-batch process. In some embodiments, the model is a metabolic model. In some embodiments, the model comprises one or more machine learning models. In some embodiments, the one or more machine learning models comprise: a neural network. In some embodiments, the method further comprises: training the neural network using data extracted from representative processes exhibiting no lactate spike and representative processes exhibiting a lactate spike. In some embodiments, the extracted data characterizes one or more of product composition, initial conditions, and nutrient additions of the fed-batch process. In some embodiments, the providing data comprises one or more: displaying the identified at least one factor in a graphical user interface, storing the identified at least one factor in physical persistence, loading the identified at least one factor in memory, or transmitting the identified at least one factor over a network to a remote computing system. In some embodiments, the identified at least one factor indicates that pyruvate concentration in one or more feeds to cells in a bioreactor in the fed-batch process needs to be reduced in order to reduce the lactate spike. In some embodiments, the identified at least one factor indicates that the concentration of one or more amino acids in one or more feeds to cells in a bioreactor in the fed-batch process needs to be increased in order to reduce the lactate spike.

In another aspect, provided herein is a method for producing a protein of interest as part of a fed-batch process comprising: receiving data characterizing the fed-batch process; initializing a model of a mammalian cell line; simulating, using the initialized model and the received data, the fed-batch process to characterize a lactate spike; identifying, based on the simulating, at least one factor contributing to the lactate spike; and modifying one or more operational parameters of the fed-batch processes based on the identified at least one factor. In some embodiments, the modifying comprises reducing pyruvate concentration in one or more feeds to cells in a bioreactor in the fed-batch process. In some embodiments, the modifying comprises: increasing one or more amino acids in one or more feeds to cells in a bioreactor in the fed-batch process.

In another aspect, provided herein is a method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding a volume of a first feed (e.g., a first complex feed) within 0 to 6 days after initiation of the fed-batch culturing of the cells, wherein the first feed comprises a first pyruvate concentration, and wherein the first pyruvate concentration is about 65% to about 100% lower than the pyruvate concentration used in same volume of the first feed in a second fed-batch culturing of the cells under the same conditions where a lactate spike is observed. In some embodiments, the volume of the first feed is within 0 to 3 days. In some embodiments, the volume of the first feed is within 3 to 5 days. In some embodiments, the volume of the first feed is within 3 to 6 days. In some embodiments, the volume of the first feed is within 4 to 6 days. In some embodiments, the volume of the first feed is within 5 to 6 days. In some embodiments, the concentration of one or more amino acids is increased. In some embodiments, the increase in the concentration of one or more amino acids is about 0% to about 100%. In some embodiments, the one or more amino acids is selected from the group consisting of glutamate, valine, leucine, threonine, aspartate, and isoleucine, or a combination thereof. In some embodiments, the one or more amino acids are selected from the group consisting of glutamate, valine, and leucine, or a combination thereof. In some embodiments, the one or more amino acids are glutamate, valine, or a combination thereof. In some embodiments, the cells are CHO cells. In specific embodiments, the protein of interest is a recombinant protein. In some embodiments, the protein of interest is an antibody, a cytokine, an antigen, an enzyme, or a coagulant. In some embodiments, the protein of interest is an antibody, a cytokine, or an antigen. In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the cytokine is IL-12, IL-23, IL-1β, IL-6, IL-15, IL-2, IL-5, TNF-alpha, IL-9, or IL-17.

In another aspect, provided herein is a method for identifying the pyruvate concentration to use in one or more feeds in a fed-batch process, comprising: (a) fed-batch culturing cells comprising a nucleic acid encoding a protein of interest in a first bioreactor under conditions sufficient for the cells to produce the protein, wherein the fed-batch culturing comprises adding a volume of one or more feeds (e.g., one or more complex feeds) comprising a first pyruvate concentration; (b) fed-batch culturing the same cells in a second bioreactor under the same conditions used in the fed-batch culturing in step (a), except that the one or more feeds comprises a second pyruvate concentration, wherein the second pyruvate concentration is about 65% to about 100% (e.g., about 65% to about 75%, about 75% to about 85%, about 85% to about 95%, about 80% to about 95%, or about 85% to about 100%) lower than the first pyruvate concentration; (c) measuring lactate concentration in the fed-batch culturing in step (a) within about 12 to about 72 hours (e.g., about 12 hours, about 18 hours, about 24 hours, about 48 hours, or about 72 hours) after each feed and measuring lactate concentration in the fed-batch culturing in step (b) within about 12 to about 72 hours (e.g., about 12 hours, about 18 hours, about 24 hours, about 48 hours, or about 72 hours) after each feed; and (d) comparing the lactate concentration measured for the fed-batch culturing in step (a) to the lactate concentration measured for the fed-batch culturing in step (b), wherein a decrease in the lactate concentration for the fed-batch culturing in step (b) relative to the lactate concentration for the fed-batch culturing in step (a) indicates that the pyruvate concentration used in the one or more feeds in the fed-batching culturing in step (b) are better for fed-batch culturing the cells in a bioreactor. In some embodiments, the method further comprises (e) implementing manufacture of the protein by fed-batch culturing of the cells comprising said nucleic acid under conditions sufficient for the cells to produce the recombinant protein, wherein the fed-batch culturing comprises adding a volume of one or more feeds comprising the second pyruvate concentration. In some embodiments, the cells are CHO cells. In specific embodiments, the protein of interest is a recombinant protein. In some embodiments, the protein of interest is an antibody, a cytokine, an antigen, an enzyme, or a coagulant. In some embodiments, the protein of interest is an antibody, a cytokine, or an antigen. In some embodiments, the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin. In some embodiments, the antibody binds to an antigen of a pathogen. In some embodiments, the pathogen is a virus, a bacteria, a fungus, or a parasite. In some embodiments, the cytokine is IL-12, IL-23, IL-1β, IL-6, IL-15, IL-2, IL-5, TNF-alpha, IL-9, or IL-17. See Section 4.1.1 for additional information and embodiments regarding proteins of interest.

In another aspect, provided herein is a method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding one or more feeds (e.g., one or more complex feeds) during expansion of the cells and reducing the concentration of pyruvate in one or more additional feeds (e.g., one or more additional complex feeds) before peak cell density, wherein the pyruvate concentration in the one or more additional feeds is reduced by 65% to 100% relative to the pyruvate concentration in the one or more feeds during expansion of the cells. In some embodiments, the peak cell density is at about 6 to about 7 days in the fed-batch culturing of the cells and the reduction in pyruvate concentration in the one or more additional feeds is at about days 3 to 5 (e.g., at about day 3, about day 4, or about 5 day) in the fed-batch culturing of the cells. In some embodiments, the concentration of one or more amino acids in the one or more additional feeds is increased before peak cell density. In some embodiments, the concentration of one or more amino acids in the one or more additional feeds is increased by 0% to 100% relative to the concentration of the one or more amino acids in the one or more feeds during expansion of the cells and before peak cell density. In some embodiments, the one or more amino acids are selected from the group consisting of glutamate, valine, leucine, threonine, aspartate, and isoleucine, or a combination thereof. In some embodiments, the one or more amino acids are selected from the group consisting of glutamate, valine, and leucine, or a combination thereof. In some embodiments, the one or more amino acids are glutamate, valine, or a combination thereof. In some embodiments, the cells are CHO cells.

### 3. BRIEF DESCRIPTION OF THE DRAWINGS

FIG. 1. Bioreactor for fed-batch process for producing Antibody A exhibits lactate spike, increased glucose consumption during lactate spike, depressed glucose consumption following lactate spike, and increased osmolality due to base addition. Differences in viable cell density, viability, and glutamine concentration were not observed.

FIG. 2. Bioreactors for fed-batch process for producing Antibody B and Antibody C exhibit lactate spike. Differences in viable cell density were not observed.

FIG. 3. Lactate production and glucose consumption per cell in bioreactors for fed-batch processes for producing Antibody A, Antibody B, and Antibody C.

FIGS. 4A-4C. Viable cell density (FIG. 4A), titer (FIG. 4B), and lactate (FIG. 4C) for cell culture processes operated under equivalent conditions displaying significant sudden lactate accumulation (solid lines), moderate sudden lactate accumulation (dashed dotted lines), or no sudden lactate accumulation (dashed lines) with no apparent growth or titer differences.

FIGS. 5A-5B. Schematic of Digital Twin (i.e., model). FIG. 5A) The digital twin includes three separate models that together describe the dynamics of a cell culture process. FIG. 5B) Individual models of DT are mapped to a physical system where cells (circle), extracellular metabolites (square) and volume manipulations (arrow) correspond with their respective models in FIG. 5A (i.e. kinetic cell model, extracellular reaction model, and reactor model, respectively).

FIG. 6. Two representative processes displaying ‘no lactate spike’ (M19L059) or a ‘lactate spike’ (M19L062) were used to train a metabolic model. Updated model parameters resulted in accurate fitting of the model (solid lines) to the process data (dots) for both phenotypes.

FIG. 7. Sensitivity analysis of feed media components on lactate formation. Positive correlation (outlined with dashed lines)=increasing concentration increases lactate formation. Negative correlation (outlined with solid lines)=increasing concentration decreases lactate formation.

FIG. 8. Diagram of TCA cycle entry points for media components most likely to affect lactate formation. Components from sensitivity analysis are underlined. Dashed lines indicate multiple reactions prior to entry points.

FIGS. 9A-9B. Experimental verification results of model-identified levers. FIG. 9A) Lactate concentration compared to previous lactate spike processes (dash-dotted lines). Conditions with lower pyruvate (thin solid lines) produced consistently lower lactate concentration. FIG. 9B) Normalized titer results. Each condition (n≥2) was normalized to average titer for lactate spike processes shown in FIG. 4C. Error bars represents standard deviation.

FIGS. 10A-10B. The Digital Twin calculated the flux distributions at 120 hr (time of lactate spike) in μmol/cell/day. FIG. 10A) Glycolytic flux for high lactate process where 55% of glucose is converted to lactate. FIG. 10B) Low pyruvate glycolytic flux where 22% of glucose is converted to lactate and majority of carbon enters TCA cycle.

FIG. 11. Proposed mechanism of lactate spike and its mitigation. 1) High concentrations of extracellular pyruvate activate the uptake of glucose. 2) The high lactate concentration allosterically inhibits pfk thus reducing glycolysis fluxes.

FIG. 12. Experimental conditions for verification of sensitivity analysis. As described, the experimental conditions refers to those described in FIG. 9B.

### 4. DESCRIPTION

### 4.1 Fed-Batch Process Methods

The present disclosure is directed, in part, to methods, systems, and compositions for reducing lactate accumulation during a fed-batch process for production of a protein using a genome-based metabolic model. For example, the present disclosure addresses the phenomenon of rapid lactate accumulation in the middle of a fed-batch process for production of a protein of interest (referred to herein as a “lactate spike”). In particular, during fed-batch production of a protein of interest, it was observed that cells sporadically produced 2-3 g/L of lactate over 24 hours near peak cell density followed by consumption of the excess lactate over the remainder of the fed-batch process. When this phenomenon occurred, a reduction in glucose metabolism, a reduction in product expression, and lower product quality were observed. In particular, the following was observed with the lactate spike phenomenon: (1) increased glucose consumption during lactate spike followed by depressed glucose consumption; (2) decreased oxygen consumption; and (3) increased culture osmolality due to base addition. The inventors surprisingly found that low sodium pyruvate in the complex feed eliminated the lactate spike, while, e.g., improving the titers of the protein of interest.

In specific embodiments, the methods described herein for fed-batch processes for production of a protein of interest are environmentally friendly since the methods may improve the titer of protein produced and thus, reduce the number of processes needed to be run to achieve the amount of protein desired.

As used herein, the terms “about” and “approximately” mean within 20%, within 15%, within 10%, within 9%, within 8%, within 7%, within 6%, within 5%, within 4%, within 3%, within 2%, within 1%, or less of a given value or range.

Fed-batch processes are generally known in the art and employed to optimize protein production (see, e.g., Y. M. Huang et al., Biotechnol Prog. 2010 September-October; 26 (5): 1400-10). As used herein, the terms “fed-batch process”, “fed-batch culture” and analogous terms are used to refer to a technique for cultivating cells (e.g., a mammalian cell line) in a bioreactor. Generally, a fed-batch process is a biotechnological process where one or more nutrients are fed to a bioreactor discretely or continuously during cultivation of cells (e.g., mammalian cell line) and in which a protein of interest remains in the bioreactor until the end of the run. A fed-batch culture is usually terminated at some point and the cells and/or components in the medium are harvested and optionally purified.

The fed-batch process typically involves a growth phase and a production phase. Typically, the growth phase (also known as a seed train or seed culture) is when all components for cell culturing are supplied to the bioreactor at the start of the culturing process then cell population is expanded until ready for production scale. As such, the bioreactor is inoculated with cells at a suitable seeding density for the initial cell growth phase depending on the starting cells (e.g., cell line). Typically, the production phase is when the protein is produced and harvested. See Section 4.1.5 for information and embodiments regarding purification.

In the fed-batch process, typically media is supplemented at intervals during cell culture. These feeds (e.g., complex feeds) are typically used during the production phase. The feeds may be performed at intervals at a frequency of multiple feeds per day, every day, or every 2-3 days, for the duration of the production culture. In some embodiments, the feeds may be performed at least 2 times, at least 3 times, at least 4 times, at least 5 times, at least 6 times, at least 7 times, or at least 8 times, throughout the duration of the production culture for at least one week and up to two or more weeks culture. For example, the feeds may be performed at least 2 times, at least 3 times, at least 4 times, at least 5 times, at least 6 times, at least 7 times, or at least 8 times, throughout the duration of the production culture for 1 to 2 weeks (e.g., 1 week or 2 weeks). In another example, the feeds may be performed at least 2 times, at least 3 times, at least 4 times, at least 5 times, at least 6 times, at least 7 times, or at least 8 times, throughout the duration of the production culture for 2 to 4 weeks (e.g., 2 weeks, 3 weeks, or 4 weeks). In some embodiments, the supplemental feed could be performed on each day for the duration of the culture. Alternative culture feeding schedules are also envisioned. The feeds will generally contain nutrients that are depleted during cell culture. In some embodiments, the first feed is on days 0-3.

As used herein, term “nutrient” may refer to any compound, molecule, or substance used by an organism to live, grow, produce a protein(s) of interest, or otherwise add biomass. Examples of nutrients may include carbohydrate sources (e.g., simple sugars such as glucose, galactose, maltose or fructose, or more complex sugars), amino acids, and vitamins (e.g., B group vitamins (e.g., B12), vitamin A vitamin E, riboflavin, thiamine and biotin).

The term “amino acid” may refer to any of the twenty standard amino acids (i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid), single stereoisomers thereof, and racemic mixtures thereof. In some embodiments, the term “amino acid” refers to any of the twenty standard amino acids or a stereoisomer thereof. In some embodiments, the amino acid is a single stereoisomer (e.g., an L-stereoisomer) of any of the twenty standard amino acids. The term “amino acid” can also refer to the known non-standard amino acids, e.g., 4-hydroxyproline, hydroxy-proline, s-sulfocysteine, phosphotyrosine, e-N,N,N-trimethyllysine, 3-methylhistidine, 5-hydroxylysine, O-phosphoserine, g-carboxyglutamate, e-N-acetyllysine, co-N-methylarginine, N-acetylserine, N,N,N-trimethylalanine, N-formylmethionine, g-aminobutyric acid, histamine, dopamine, thyroxine, citrulline, ornithine, b-cyanoalanine, homocysteine, azaserine, and S-adenosylmethionine.

As used herein, the term “pyruvate” includes the free form of pyruvic acid as well as acid salts, including sodium pyruvate and other acid salts. In a specific embodiment, the pyruvate is sodium pyruvate.

In one aspect, provided herein is a method of producing a protein(s) of interest, the method comprising: (a) fed-batch culturing cells comprising a nucleic acid encoding the protein in a bioreactor under conditions sufficient for the cells to produce the protein, wherein the fed-batch culturing comprises adding one or more feeds (e.g., 1, 2, 3, 4 or more feeds (e.g., complex feeds)) comprising a pyruvate concentration that is reduced by about 65% to about 100%; and (b) purifying the protein from the cells or the liquid culture medium. In some embodiments, the pyruvate concentration is reduced by at least 65%, at least 70%, at least 75%, at least 80%, or at least 85%. In some embodiments, the pyruvate concentration is reduced by at least 90%, at least 95%, or at least 98%. In some embodiments, the pyruvate concentration is reduced by about 65% to about 80%, about 70% to about 80%, about 75% to about 85%, or about 80% to about 95%. In specific embodiments, the reduction in pyruvate concentration is relative to the pyruvate concentration used is a fed-batch process that is the same except for the pyruvate concentration and in which a lactate spike is observed. In specific embodiments, the reduction in pyruvate concentration is relative to the pyruvate concentration used is a fed-batch process that is the same except for the pyruvate concentration and/or the concentration of one or more amino acids and in which a lactate spike is observed. In some embodiments, the concentration of one or more amino acids in the one or more feeds (e.g., one or more complex feeds) is increased by 0% to about 100%. In some embodiments, the concentration of one or more amino acids in the one or more feeds is increased by 0% to 20%, 20% to 40%, 40% to 60%, 60% to 80%, or 80% to 100%. In some embodiments, the concentration of one or more amino acids in one or more of the feeds is increased by about 5% to about 25%, about 25% to about 50%, about 50% to about 75%, or about 75% to about 95%. In some embodiments, concentration of more amino acids in the one or more feeds is increased by 0% to 40%. In some embodiments, the concentration of one or more amino acids in the one or more feeds is increased by at least 5%, at least 10%, at least 20%, at least 30%, or at least 40%. In some embodiments, the concentration of one or more amino acids in the one or more feeds is increased by at least 50%, at least 60%, at least 70%, at least 80%, at least 85%, at least 90%, or at least 95%. In some embodiments, the one or more amino acids are selected from the group consisting of glutamate, valine, leucine, threonine, aspartate, and isoleucine, or a combination thereof. In some embodiments, the one or more amino acids are selected from the group consisting of glutamate, valine, and leucine, or a combination thereof. In some embodiments, the one or more amino acids are glutamate, valine, or a combination thereof.

In some embodiments, the concentration of one or more amino acids is reduced at least 5%, at least 10%, at least 15%, at least 20%, or at least 25%. In some embodiments, the concentration of one or more amino acids is reduced by at least 30%, at least 35%, at least 40%, at least 45%, at least 50%, at least 55%, or at least 60%. In some embodiments, the concentration of one or more amino acids is reduced by at least 65%, at least 70%, at least 75%, at least 80%, or at least 85%. In some embodiments, the pyruvate concentration is reduced by at least 90%, at least 95%, at least 98%, at least 99%, or 100%. In some embodiments, the concentration of one or more amino acids is reduced by about 5% to about 10%, about 10% to about 25%, about 20% to about 40%, about 25% to about 50%, about 40% to 60%, or about 50% to about 65%. In some embodiments, the concentration of one or more amino acids is reduced by about 65% to about 80%, about 70% to about 80%, about 75% to about 85%, about 80% to about 95%, or about 90% to about 100%. In some embodiments, the one or more amino acids reduced is asparagine. The volume of a feed (e.g., complex feed) may be 0.1 mL to about 1 mL, about 1 mL to about 20 mL, about 20 mL to about 50 mL, about 50 mL to about 100 mL, about 100 mL to about 500 mL, or about 500 mL to about 1000 mL. A feed (e.g., complex feed) may have a volume of about 1 L to about 10 L, about 10 L to about 50 L, about 50 L to about 100 L, about 100 L to about 500 L, or about 500 L to 2000 L. One skilled in the art may appreciate that the volume of a feed (e.g., complex feed) may be in the range of 0.05% to 10% of the bioreactor working volume.

In one aspect, provided herein is a method for reducing/mitigating a lactate spike in a fed-batch process for producing a protein(s) of interest, comprising reducing pyruvate (e.g., sodium pyruvate) concentration in one or more feeds (e.g., one or more complex feeds) to cells in a bioreactor in the fed-batch process. In some embodiments, the concentration of pyruvate (e.g., sodium pyruvate) in the one or more feeds is reduced by about 10% to about 30% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 25% to about 50% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 25% to about 75% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 50% to about 75% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 65% to about 100% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 75% to about 95% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 85% to about 98% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 10%, about 15%, about 25%, about 30%, about 35%, or about 40% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 45%, about 50%, about 55%, about 60%, about 65%, or about 70% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 75%, about 80%, about 85%, about 90%, about 95%, about 98%, about 99%, or about 100% relative to the concentration of pyruvate (e.g., sodium pyruvate) found in the one or more feeds in the same fed-batch process where a lactate spike is observed.

In some embodiments, in addition to reducing the concentration of pyruvate (e.g., sodium pyruvate) in one or more feeds (e.g., one or more complex feeds) of the fed-batch process for producing a protein of interest, the one or more feeds comprise an increased concentration of one or more amino acids. In some embodiments, the one or more amino acids are selected from the group consisting of glutamate, valine, leucine, aspartate, isoleucine, and threonine, or a combination thereof. In some embodiments, the one or more amino acids are selected from the group consisting of glutamate, valine, leucine, aspartate, isoleucine, and threonine. In some embodiments, the one or more amino acids are glutamate, valine, and leucine. In some embodiments, the one or more amino acids are glutamate and valine. In some embodiments, the increase in concentration of an amino acid is by about 0% to about 100% relative to the concentration of the amino acid found in the one or more feeds in the same fed-batch process where a lactate spike is observed. In some embodiments, the increase in concentration of an amino acid is by about 0.5% to about 10% relative to the concentration of the amino acid found in the one or more feeds in the same fed-batch process where a lactate spike is observed. In some embodiments, the increase in concentration of an amino acid is by about 0.5%, about 1%, about 2%, about 3%, about 4% or about 5% relative to the concentration of the amino acid found in the one or more feeds in the same fed-batch process where a lactate spike is observed. In some embodiments, the increase in concentration of an amino acid is by about 6%, about 7%, about 8%, about 9%, or about 10% relative to the concentration of the amino acid found in the one or more feeds in the same fed-batch process where a lactate spike is observed. In some embodiments, the increase in concentration of an amino acid is by about 0.5% to about 100% relative to the concentration of the amino acid found in the one or more feeds in the same fed-batch process where a lactate spike is observed. In some embodiments, the increase in concentration of an amino acid is by about 5% to about 100%, about 5% to about 75%, about 5% to about 50%, or about 5% to about 25% relative to the concentration of the amino acid found in the one or more feeds in the same fed-batch process where a lactate spike is observed. In some embodiments, the increase in concentration of an amino acid is by about 25% to about 100%, about 25% to about 75%, or about 25% to about 50% relative to the concentration of the amino acid found in the one or more feeds in the same fed-batch process where a lactate spike is observed. In some embodiments, the increase in concentration of an amino acid is by about 0% to about 20%, about 20% to about 40%, about 40% to about 60%, or about 20% to about 60% relative to the concentration of the amino acid found in the one or more feeds in the same fed-batch process where a lactate spike is observed. In some embodiments, the increase in concentration of an amino acid is by about 0% to about 40% about 1% to about 40%, about 60% to about 80%, or about 80% to about 100% relative to the concentration of the amino acid found in the one or more feeds in the same fed-batch process where a lactate spike is observed. In some embodiments, the increase in concentration of an amino acid is by at least 0.05%, at least 1% at least 5%, at least 10%, at least 20%, at least 20%, at least 25%, at least 30%, at least 35% or at least 40% relative to the concentration of the amino acid found in the one or more feeds in the same fed-batch process where a lactate spike is observed. In some embodiments, the increase in concentration of an amino acid is by at least 45%, at least 50% at least 55%, at least 60%, at least 65%, at least 70%, at least 75%, or at least 80% relative to the concentration of the amino acid found in the one or more feeds in the same fed-batch process where a lactate spike is observed. In some embodiments, the increase in concentration of an amino acid is by at least 85%, at least 90% at least 95%, at least 98%, at least 99%, or 100% relative to the concentration of the amino acid found in the one or more feeds in the same fed-batch process where a lactate spike is observed.

In specific embodiments, the lactate spike is mitigated by feed addition (e.g., complex feed addition). In some embodiments, the lactate spike is mitigated by feed addition (e.g., complex feed addition) between culture days five to six. In some embodiments, the lactate spike is mitigated by feed addition (e.g., complex feed addition) between culture days four to seven. In some embodiments, the lactate spike is mitigated by feed addition (e.g., complex feed addition) between culture days three to eight. In some embodiments, the lactate spike is mitigated by feed addition (e.g., complex feed addition) between culture days two to ten. In some embodiments, the lactate spike is mitigated by feed addition (e.g., complex feed addition) between culture days four to five. In some embodiments, the lactate spike is mitigated by feed addition (e.g., complex feed addition) between culture days six to seven. In some embodiments, the lactate spike is mitigated by feed addition (e.g., complex feed addition) between culture days seven to eight. In some embodiments, the lactate spike is mitigated by complex feed addition between culture days eight to nine. In some embodiments, the lactate spike is mitigated by feed addition (e.g., complex feed addition) between culture days nine to ten. In some embodiments, the lactate spike is mitigated by feed addition (e.g., complex feed addition) on day three of the culture. In some embodiments, the lactate spike is mitigated by complex feed addition on day four of the culture. In some embodiments, the lactate spike is mitigated by feed addition (e.g., complex feed addition) on day five of the culture. In some embodiments, the lactate spike is mitigated by feed addition (e.g., complex feed addition) on day six of the culture. In some embodiments, the lactate spike is mitigated by complex feed addition on day seven of the culture. In some embodiments, the lactate spike is mitigated by feed addition (e.g., complex feed addition) on day eight of the culture.

In some embodiments, the cells are mammalian cells. Non-limiting examples of mammalian host cell lines suitable for use in the present disclosure include the Chinese hamster ovary (CHO), mouse myeloma derived NSO and Sp2/0 cells, human embryonic kidney cells (HEK293), and human embryonic retinal-derived PER.C6 cells. In some embodiments, the mammalian cell line is a CHO cell line. See Section 4.1.2 for additional information and embodiments regarding cells.

In specific embodiments, the media and feeds (e.g., complex feeds) used in the fed-batch process is appropriate for the cells being used. In specific embodiments, the media and feeds (e.g., complex feeds) used in the fed-batch process allows the cells being used to grow and produce the protein(s) of interest. See Section 4.1.4 for additional information and embodiments regarding media and feeds.

The bioreactor used for the fed-batch has a suitable volume that allows for the cultivation and propagation of biological cells capable of producing the protein(s) of interest. For example, the volume of the bioreactor can be about 10 milliliters (mL) to about 25,000 L. In another example, the volume of the bioreactor can be about 0.5 liters (L) to about 25,000 L. In some embodiments, the volume of the bioreactor can be about 10 mL. In some embodiments, the volume of the bioreactor can be about 10 mL about 100 mL. In some embodiments, the volume of the bioreactor can be about 100 mL to about 200 mL. In some embodiments, the volume of the bioreactor can be about 100 mL to about 300 mL. In some embodiments, the volume of the bioreactor can be about 100 mL to about 500 mL. In some embodiments, the volume of the bioreactor can be about 500 mL to about 750 mL. In some embodiments, the volume of the bioreactor can be about 500 mL to about 1000 mL. In some embodiments, the volume of the bioreactor can be about 500 mL to about 2 L. In some embodiments, the volume of the bioreactor can be about 1 L to about 5 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 3 L. In some embodiments, the volume of the bioreactor can be less than or equal to about 5 L. In some embodiments, the volume of the bioreactor can be less than or equal to about 10 L. In some embodiments, the volume of the bioreactor can be less than or equal to about 25 L. In some embodiments, the volume of the bioreactor can be less than or equal to about 50 L. In some embodiments, the volume of the bioreactor can be less than or equal to about 100 L. In some embodiments, the volume of the bioreactor can be less than or equal to about 250 L. In some embodiments, the volume of the bioreactor can equal to or above 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be less than or equal to about 5,000 L. In some embodiments, the volume of the bioreactor can be less than or equal to about 10,000 L. In some embodiments, the volume of the bioreactor can be less than or equal to about 15,000 L. In some embodiments, the volume of the bioreactor can be less than or equal to about 25,000 L. See Section 4.1.3 for additional information and embodiments regarding bioreactors.

In some embodiments, the lactate spike occurs between culture days five to six. In some embodiments, the lactate spike occurs between culture days four to seven. In some embodiments, the lactate spike occurs between culture days three to eight. In some embodiments, the lactate spike occurs between culture days two to ten. In some embodiments, the lactate spike occurs between culture days four to five. In some embodiments, the lactate spike occurs between culture days six to seven. In some embodiments, the lactate spike occurs between culture days seven to eight. In some embodiments, the lactate spike occurs between culture days eight to nine. In some embodiments, the lactate spike occurs between culture days nine to ten. In some embodiments, the lactate spike occurs on day three of the culture. In some embodiments, the lactate spike occurs on day four of the culture. In some embodiments, the lactate spike occurs on day five of the culture. In some embodiments, the lactate spike occurs on day six of the culture. In some embodiments, the lactate spike occurs on day seven of the culture. In some embodiments, the lactate spike occurs on day eight of the culture.

In some embodiments, the lactate spike is mitigated by one or more of (a) a decrease in asparagine, pyruvate, or a combination thereof; and/or (b) an increase in glutamic acid, isoleucine, leucine, aspartic acid, valine, threonine, or a combination thereof. In some embodiments, the lactate spike is mitigated by an increase in one or more amino acids selected from the group consisting of glutamate, valine, leucine, threonine, aspartate, and isoleucine, or a combination thereof. In specific embodiments, the lactate spike is mitigated by a decrease in pyruvate. In some embodiments, the lactate spike is mitigated by an increase in glutamate, valine, leucine, or a combination thereof. In some embodiments, the lactate spike is mitigated by an increase in glutamate, valine, or a combination thereof. In certain embodiments, the lactate spike is mitigated by an increase in glutamic acid, valine, or a combination thereof. In some embodiments, the lactate spike is mitigated by an increase in glutamate, valine, and leucine, or a combination thereof. In some embodiments, the lactate spike is mitigated by an increase in glutamate, valine, or a combination thereof.

As provided herein, the mitigation of lactate spike is able to improve productivity and/or product quality (e.g., capillary isoelectric focusing (cIEF), purity, glycans). In some embodiments, productivity of the bioreactor process is increased by about 5%. In some embodiments, productivity of the bioreactor process is increased by about 10%. In some embodiments, productivity of the bioreactor process is increased by about 20%. In some embodiments, productivity of the bioreactor process is increased by about 30%. In some embodiments, productivity of the bioreactor process is increased by about 40%. In some embodiments, productivity of the bioreactor process is increased by about 50%. In some embodiments, productivity of the bioreactor process is increased by about 60%. In some embodiments, productivity of the bioreactor process is increased by about 70%. In some embodiments, productivity of the bioreactor process is increased by about 80%. In some embodiments, productivity of the bioreactor process is increased by about 90%. In some embodiments, productivity of the bioreactor process is increased by more than 100%.

In some embodiments, the product quality is increased by about 5%. In some embodiments, the product quality is increased by about 10%. In some embodiments, the product quality is increased by about 20%. In some embodiments, the product quality is increased by about 30%. In some embodiments, the product quality is increased by about 40%. In some embodiments, the product quality is increased by about 50%. In some embodiments, the product quality is increased by about 60%. In some embodiments, the product quality is increased by about 70%. In some embodiments, the product quality is increased by about 80%. In some embodiments, the product quality is increased by about 90%. In some embodiments, the product quality is increased by more than 100%.

In some embodiments, the method for reducing a lactate spike results in an increase in titer of the protein of interest. In some embodiments, the method for reducing a lactate spike results an increase of about 5% to about 30% in the titer of the protein of interest. In some embodiments, the method for reducing a lactate spike results an increase of about 5% to about 20% in the titer of the protein of interest. In some embodiments, the method for reducing a lactate spike results an increase of about 5% to about 15% in the titer of the protein of interest. In some embodiments, the method for reducing a lactate spike results an increase of about 5% to about 10% in the titer of the protein of interest. In some embodiments, the method for reducing a lactate spike results an increase of about 10% to about 30% in the titer of the protein of interest. In some embodiments, the method for reducing a lactate spike results an increase of about 10% to about 20% in the titer of the protein of interest. In some embodiments, the method for reducing a lactate spike results an increase of about 20% to about 30% in the titer of the protein of interest. In some embodiments, the method for reducing a lactate spike results an increase of about 5%, about 10%, about 15%, about 20%, about 25%, or about 30% in the titer of the protein of interest. In some embodiments, the method for reducing a lactate spike results an increase of about 25% to about 75% in the titer of the protein of interest. In some embodiments, the method for reducing a lactate spike results an increase of about 50% to about 75% in the titer of the protein of interest. In some embodiments, the method for reducing a lactate spike results an increase of about 75% to about 95% in the titer of the protein of interest.

In some embodiments, the increase in titer is an increase of about 10%. In some embodiments, the increase in titer is an increase of about 20%. In some embodiments, the increase in titer is an increase of about 30%. In some embodiments, the increase in titer is an increase of about 40%. In some embodiments, the increase in titer is an increase of about 50%. In some embodiments, the increase in titer is an increase of about 60%. In some embodiments, the increase in titer is an increase of about 70%. In some embodiments, the increase in titer is an increase of about 80%. In some embodiments, the increase in titer is an increase of about 90%. In some embodiments, the increase in titer is an increase of about 100%. In some embodiments, the increase in titer is an increase of more than 100%.

In some embodiments, the protein production yield or titer, which can be expressed in grams of protein product per liter of culture medium, from cells cultured according of the disclosure is at least 100 mg/L, at least 1 g/L, at least 1.2 g/L, at least 1.4 g/L, at least 1.6 g/L, at least 1.8 g/L, at least 2 g/L, at least 2.5 g/L, at least 3 g/L, at least, 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 15 g/L, or at least 20 g/L.

In specific embodiments, a reduction in pyruvate (e.g., sodium pyruvate) increases the titer by about 0.5 g/L. In specific embodiments, a reduction in pyruvate (e.g., sodium pyruvate) increases the titer by about 1 g/L. In specific embodiments, a reduction in pyruvate (e.g., sodium pyruvate) increases the titer by about 1.5 g/L. In specific embodiments, a reduction in pyruvate (e.g., sodium pyruvate) increases the titer by about 2 g/L. In specific embodiments, a reduction in pyruvate (e.g., sodium pyruvate) increases the titer by about 2.5 g/L. In specific embodiments, a reduction in pyruvate (e.g., sodium pyruvate) increases the titer by about 3 g/L. In specific embodiments, a reduction in pyruvate (e.g., sodium pyruvate) increases the titer by 3.5 g/L. In specific embodiments, a reduction in pyruvate (e.g., sodium pyruvate) increases the titer by about 4 g/L. In specific embodiments, a reduction in pyruvate (e.g., sodium pyruvate) increases the titer by 4.5 g/L. In specific embodiments, a reduction in pyruvate (e.g., sodium pyruvate) increases the titer by about 5 g/L. In specific embodiments, a reduction in pyruvate (e.g., sodium pyruvate) increases the titer by 5.5 g/L. In specific embodiments, a reduction in pyruvate (e.g., sodium pyruvate) increases the titer by about 6 g/L. In some embodiments, a reduction in pyruvate (e.g., sodium pyruvate) increases the titer by about 0.5 g/L to about 6 g/L. In some embodiments, a reduction in pyruvate (e.g., sodium pyruvate) increases the titer by about 1 g/L to about 6 g/L, about 2 g/L to about 6 g/L, about 2 g/L to about 4 g/L. In some embodiments, a reduction in pyruvate (e.g., sodium pyruvate) increases the titer by about 3 g/L to about 6 g/L, about 4 g/L to about 6 g/L, about 3 g/L to about 5 g/L, about 3 g/L to about 4 g/L, or about 4 g/L to about 5 g/L.

In some embodiments, the method for reducing a lactate spike results in an increase in viable cell density (VCD). In some embodiments, the method for reducing a lactate spike results an increase of about 5% to about 30% in VCD. In some embodiments, the method for reducing a lactate spike results an increase of about 5% to about 20% in VCD. In some embodiments, the method for reducing a lactate spike results an increase of about 5% to about 15% in VCD. In some embodiments, the method for reducing a lactate spike results an increase of about 5% to about 10% in VCD. In some embodiments, the method for reducing a lactate spike results an increase of about 10% to about 30% in VCD. In some embodiments, the method for reducing a lactate spike results an increase of about 10% to about 20% in VCD. In some embodiments, the method for reducing a lactate spike results an increase of about 20% to about 30% in VCD. In some embodiments, the method for reducing a lactate spike results an increase of about 5%, about 10%, about 15%, about 20%, about 25%, or about 30% in VCD.

In some embodiments, the method for reducing a lactate spike results in an increase in cell productivity. In some embodiments, the method for reducing a lactate spike results an increase of about 5% to about 30% in cell productivity. In some embodiments, the method for reducing a lactate spike results an increase of about 5% to about 20% in cell productivity. In some embodiments, the method for reducing a lactate spike results an increase of about 5% to about 15% in cell productivity. In some embodiments, the method for reducing a lactate spike results an increase of about 5% to about 10% in cell productivity. In some embodiments, the method for reducing a lactate spike results an increase of about 10% to about 30% in cell productivity. In some embodiments, the method for reducing a lactate spike results an increase of about 10% to about 20% in cell productivity. In some embodiments, the method for reducing a lactate spike results an increase of about 20% to about 30% in cell productivity. In some embodiments, the method for reducing a lactate spike results an increase of about 5%, about 10%, about 15%, about 20%, about 25%, or about 30% in cell productivity.

In some embodiments, the method for reducing a lactate spike results in an increase in purity (e.g., cSDS purity). In some embodiments, the method for reducing a lactate spike results in an increase of about 10% to 40% in purity (e.g., cSDS purity). In some embodiments, the method for reducing a lactate spike results in an increase of about 20% to 40% in purity (e.g., cSDS purity). In some embodiments, the method for reducing a lactate spike results in an increase of about 30% to 40% in purity (e.g., cSDS purity). In some embodiments, a reduction in sodium pyruvate results in increased capillary sodium dodecyl sulfate gel electrophoresis (cSDS) purity.

In some embodiments, the method for reducing a lactate spike results in an increased flux into the tricarboxylic acid (TCA) cycle, such as, e.g., described in the Examples, infra. In some embodiments, the method for reducing a lactate spike balances the rates of glycolytic fluxes and TCA fluxes. In some embodiments, the method for reducing a lactate spike results in one, two, or more, or all of the effects described in the Examples, infra.

In some embodiments, the method for reducing a lactate spike results in a decrease in glycation. In some embodiments, the method for reducing a lactate spike results in a decrease of about 5% to about 30% in glycation. In some embodiments, the method for reducing a lactate spike results in a decrease of about 5% to about 20% in glycation. In some embodiments, the method for reducing a lactate spike results in a decrease of about 5% to about 10% in glycation. In some embodiments, the method for reducing a lactate spike results in a decrease of about 10% to about 20% in glycation. In some embodiments, the method for reducing a lactate spike results in a decrease of about 10% to about 30% in glycation. In some embodiments, the method for reducing a lactate spike results in a decrease of about 20% to about 30% in glycation.

In some embodiments, a reduction in pyruvate (e.g., sodium pyruvate) results in improved glycation and capillary isoelectric focusing (cIEF) profile. In some embodiments, a reduction in pyruvate (e.g., sodium pyruvate) results in increased glycan complexity. For example, as provided herein, a reduction in pyruvate (e.g., sodium pyruvate) can lower G0F/G0F-GlcNac and increase G1F/G2F than relative to high pyruvate (e.g., sodium pyruvate) conditions.

In one aspect, as provided herein, the reduction in the lactate spike increases the titer of the bioreactor culture. In some embodiments, the increase in titer is an increase of about 5%. In some embodiments, the increase in titer is an increase of about 10%. In some embodiments, the increase in titer is an increase of about 20%. In some embodiments, the increase in titer is an increase of about 30%. In some embodiments, the increase in titer is an increase of about 40%. In some embodiments, the increase in titer is an increase of about 50%. In some embodiments, the increase in titer is an increase of about 60%. In some embodiments, the increase in titer is an increase of about 70%. In some embodiments, the increase in titer is an increase of about 80%. In some embodiments, the increase in titer is an increase of about 90%. In some embodiments, the increase in titer is an increase of about 100%. In some embodiments, the increase in titer is an increase of more than 100%.

In some embodiments, the protein production yield or titer, which can be expressed in grams of protein product per liter of culture medium, from cells cultured according of the disclosure is at least 100 mg/L, at least 1 g/L, at least 1.2 g/L, at least 1.4 g/L, at least 1.6 g/L, at least 1.8 g/L, at least 2 g/L, at least 2.5 g/L, at least 3 g/L, at least, 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 15 g/L, or at least 20 g/L.

In one aspect, as provided herein, the reduction in the lactate spike increases the yield of the bioreactor culture. In some embodiments, the increase in yield is an increase of about 5%. In some embodiments, the increase in yield is an increase of about 10%. In some embodiments, the increase in yield is an increase of about 20%. In some embodiments, the increase in yield is an increase of about 30%. In some embodiments, the increase in yield is an increase of about 40%. In some embodiments, the increase in yield is an increase of about 50%. In some embodiments, the increase in yield is an increase of about 60%. In some embodiments, the increase in yield is an increase of about 70%. In some embodiments, the increase in yield is an increase of about 80%. In some embodiments, the increase in yield is an increase of about 90%. In some embodiments, the increase in yield is an increase of about 100%. In some embodiments, the increase in yield is an increase of more than 100%.

In one aspect, as provided herein, the reduction in the lactate spike increases the rate of the bioreactor culture. In some embodiments, the increase in rate is an increase of about 5%. In some embodiments, the increase in rate is an increase of about 10%. In some embodiments, the increase in rate is an increase of about 20%. In some embodiments, the increase in rate is an increase of about 30%. In some embodiments, the increase in rate is an increase of about 40%. In some embodiments, the increase in rate is an increase of about 50%. In some embodiments, the increase in rate is an increase of about 60%. In some embodiments, the increase in rate is an increase of about 70%. In some embodiments, the increase in rate is an increase of about 80%. In some embodiments, the increase in rate is an increase of about 90%. In some embodiments, the increase in rate is an increase of about 100%. In some embodiments, the increase in rate is an increase of more than 100%.

The protein of interest can be any protein that one of skill in the art desires to produce using a fed-batch process. In a specific embodiment, the protein of interest may be any protein capable of being recombinantly expressed (e.g., any recombinant protein). In some embodiments, the protein of interest is an antibody or antigen-binding fragment thereof. The antibody may be monospecific, bispecific, or multi-specific (e.g., trispecific). In some embodiments, the protein of interest is an antibody. The antibody may be monoparatopic, biparatopic, or multiparatopic. The antibody may be an antibody fusion protein. The antibody or antigen binding fragment thereof may bind to any antigen (e.g., an infectious disease antigen, a cancer antigen, or an antigen associated with another disease or disorder). In some embodiments, the protein of interest is a cytokine. In some embodiments, the protein of interest is an antigen. In specific embodiments, the protein of interest is a secreted protein. In some embodiments, the protein of interest is an enzyme (e.g., human N-acetylgalactosamine-6-sulfatase (rhGALNS)), or glucocerebrosidase). The enzyme may be one used in the food industry, such as, e.g., an enzyme used in dairy, bakery, brewing, or wine making, as emulsifier, or in sweetener production. The enzyme may be used in the pharmaceutical industry, such as, e.g., a recombinant enzyme used as a biocatalysts for the preparation of chiral drug intermediates, or as a recombinant enzyme drug (e.g., a recombinant enzyme used in enzyme replacement). In some embodiments, the protein of interest is an enzyme used for enzyme replacement, such as a lysosomal storage disease enzyme. In some embodiments, the protein of interest is Agalsidase beta, Agalsidase alfa, Imiglucerase, Taliglucerase alfa, Velaglucerase alfa, Alglucerase, Sebelipase alpha, Laronidase, Idursulfase, Elosulfase alpha, Galsulfase, or Alglucosidase alpha. The protein of interest may be tissue plasminogen activator (tpa), an interferon (e.g., IFN-alpha, IFN-beta, or IFN-gamma), or an anti-nerve agent (e.g., recombinant human butyrylcholinesterase to protect against organophosphate poisoning). In some embodiments, the protein of interest is a membrane-bound protein. In some embodiments, the protein of interest is an intracellularly expressed protein. See Section 4.1.1 for additional information and embodiments regarding proteins of interest.

In specific embodiments, the media and feeds used in the fed-batch process is appropriate for the cells being used. In specific embodiments, the media and feeds used in the fed-batch process allows the cells being used to grow and produce the protein(s) of interest. See Section 4.1.4 for additional information and embodiments regarding media and feeds.

In another aspect, provided herein is a method for increasing the titer of a protein of interest in a fed-batch process for producing the protein, comprising reducing pyruvate (e.g., sodium pyruvate) concentration in one or more feeds (e.g., one or more complex feeds) to cells in a bioreactor in the fed-batch process. In some embodiments, the concentration of pyruvate (e.g., sodium pyruvate) in the one or more feeds is reduced by about 10% to about 30% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 25% to about 50% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 25% to about 75% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 50% to about 75% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 75% to about 95% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 85% to about 98% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 10%, about 15%, about 25%, about 30%, about 35%, or about 40% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 45%, about 50%, about 55%, about 60%, about 65%, or about 70% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 75%, about 80%, about 85%, about 90%, about 95%, about 98%, about 99%, or 100% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by at least 65%, at least 70%, at least 75%, at least 80%, or at least 85% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by at least 90%, at least 95%, or at least 98% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 65% to about 80%, about 70% to about 80%, about 75% to about 85%, about 80% to about 95%, or about 65% to about 100% relative to the concentration of pyruvate (e.g., sodium pyruvate) found in the one or more feeds in the same fed-batch process where a lactate spike is observed. In some embodiments, the concentration of pyruvate (e.g., sodium pyruvate) in the one or more feeds is reduced by about 65% to about 70%, about 70% to about 75%, about 75% to about 80%, about 80% to about 85%, about 90% to about 95%, or about 95% to about 100% relative to the concentration of pyruvate (e.g., sodium pyruvate) found in the one or more feeds in the same fed-batch process where a lactate spike is observed.

In some embodiments, in addition to reducing the concentration of pyruvate (e.g., sodium pyruvate) in one or more feeds (e.g., one or more complex feeds) of the fed-batch process for producing a protein of interest, the one or more feeds comprise an increased concentration of one or more amino acids. In some embodiments, the one or more amino acids are selected from the group consisting of glutamate, valine, leucine, threonine, aspartate, and isoleucine, or a combination thereof. In some embodiments, the one or more amino acids are selected from the group consisting of glutamate, valine, leucine, isoleucine, and threonine, or a combination thereof. In some embodiments, the one or more amino acids are selected from the group consisting of glutamate, valine, and leucine, or a combination thereof. In some embodiments, the one or more amino acids are glutamate, valine, or a combination thereof. In some embodiments, the one or more amino acids does not include threonine. In some embodiments, the increase in concentration of an amino acid is by about 0% to about 100% or about 0.5% to about 10% relative to the concentration of the amino acid found in the one or more feeds in the same fed-batch process where a lactate spike is observed. In some embodiments, the increase in concentration of an amino acid is by about 0.5%, about 1%, about 2%, about 3%, about 4% or about 5% relative to the concentration of the amino acid found in the one or more feeds in the same fed-batch process where a lactate spike is observed. In some embodiments, the increase in concentration of an amino acid is by about 6%, about 7%, about 8%, about 9%, or about 10% relative to the concentration of the amino acid found in the one or more feeds in the same fed-batch process where a lactate spike is observed. In some embodiments, the increase in concentration of an amino acid is by about 0.5% to about 100% relative to the concentration of the amino acid found in the one or more feeds in the same fed-batch process where a lactate spike is observed. In some embodiments, the increase in concentration of an amino acid is by about 5% to about 100%, about 5% to about 75%, about 5% to about 50%, or about 5% to about 25% relative to the concentration of the amino acid found in the one or more feeds in the same fed-batch process where a lactate spike is observed. In some embodiments, the increase in concentration of an amino acid is by about 25% to about 100%, about 25% to about 75%, or about 25% to about 50% relative to the concentration of the amino acid found in the one or more feeds in the same fed-batch process where a lactate spike is observed. In some embodiments, the increase in concentration of an amino acid is by about 0% to about 20%, about 20% to about 40%, about 40% to about 60%, or about 20% to about 60% relative to the concentration of the amino acid found in the one or more feeds in the same fed-batch process where a lactate spike is observed. In some embodiments, the increase in concentration of an amino acid is by about 0% to about 40% about 1% to about 40%, about 60% to about 80%, or about 80% to about 100% relative to the concentration of the amino acid found in the one or more feeds in the same fed-batch process where a lactate spike is observed. In some embodiments, the increase in concentration of an amino acid is by about 0% to about 40% relative to the concentration of the amino acid found in the one or more feeds in the same fed-batch process where a lactate spike is observed. In some embodiments, the increase in concentration of an amino acid is by at least 0.05%, at least 1% at least 5%, at least 10%, at least 20%, at least 20%, at least 25%, at least 30%, at least 35% or at least 40% relative to the concentration of the amino acid found in the one or more feeds in the same fed-batch process where a lactate spike is observed. In some embodiments, the increase in concentration of an amino acid is by at least 45%, at least 50% at least 55%, at least 60%, at least 65%, at least 70%, at least 75%, or at least 80% relative to the concentration of the amino acid found in the one or more feeds in the same fed-batch process where a lactate spike is observed. In some embodiments, the increase in concentration of an amino acid is by at least 85%, at least 90% at least 95%, at least 98%, at least 99%, or 100% relative to the concentration of the amino acid found in the one or more feeds in the same fed-batch process where a lactate spike is observed. See Section 4.1.1 for additional information and embodiments regarding the proteins of interest.

In some embodiments, the cells are mammalian cells. In specific embodiments, the cells are mammalian cell lines. Non-limiting examples of mammalian host cell lines suitable for use in the present disclosure include the Chinese hamster ovary (CHO), mouse myeloma derived NSO and Sp2/0 cells, human embryonic kidney cells (HEK293), and human embryonic retinoblast-derived PER.C6 cells. In some embodiments, the mammalian cell line is a CHO cell line. See Section 4.1.2 for additional information and embodiments regarding cells.

In specific embodiments, the media and feeds (e.g., complex feeds) used in the fed-batch process is appropriate for the cells being used. In specific embodiments, the media and feeds (e.g., complex feeds) used in the fed-batch process allows the cells being used to grow and produce the protein(s) of interest. See Section 4.1.4 for additional information and embodiments regarding media and feeds.

The bioreactor used for the fed-batch has a suitable volume that allows for the cultivation and propagation of biological cells capable of producing the protein(s) of interest. For example, the volume of the bioreactor can be about 10 milliliters (mL) to about 25,000 L. In another example, the volume of the bioreactor can be about 0.5 liters (L) to about 25,000 L. In some embodiments, the volume of the bioreactor can be about 10 mL. In some embodiments, the volume of the bioreactor can be about 10 mL about 100 mL. In some embodiments, the volume of the bioreactor can be about 100 mL to about 200 mL. In some embodiments, the volume of the bioreactor can be about 100 mL to about 300 mL. In some embodiments, the volume of the bioreactor can be about 100 mL to about 500 mL. In some embodiments, the volume of the bioreactor can be about 500 mL to about 750 mL. In some embodiments, the volume of the bioreactor can be about 500 mL to about 1000 mL. In some embodiments, the volume of the bioreactor can be about 500 mL to about 2 L. In some embodiments, the volume of the bioreactor can be about 1 L to about 5 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 3 L. In some embodiments, the volume of the bioreactor can be less than or equal to about 5 L. In some embodiments, the volume of the bioreactor can be less than or equal to about 10 L. In some embodiments, the volume of the bioreactor can be less than or equal to about 25 L. In some embodiments, the volume of the bioreactor can be less than or equal to about 50 L. In some embodiments, the volume of the bioreactor can be less than or equal to about 100 L. In some embodiments, the volume of the bioreactor can be less than or equal to about 250 L. In some embodiments, the volume of the bioreactor can equal to or above 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be less than or equal to about 5,000 L. In some embodiments, the volume of the bioreactor can be less than or equal to about 10,000 L. In some embodiments, the volume of the bioreactor can be less than or equal to about 15,000 L. In some embodiments, the volume of the bioreactor can be less than or equal to about 25,000 L. See Section 4.1.3 for additional information and embodiments regarding bioreactors.

In some embodiments, the method results an increase of about 5% to about 30% in the titer of the protein of interest. In some embodiments, the method results an increase of about 5% to about 20% in the titer of the protein of interest. In some embodiments, the method results an increase of about 5% to about 15% in the titer of the protein of interest. In some embodiments, the method results an increase of about 5% to about 10% in the titer of the protein of interest. In some embodiments, the method results an increase of about 10% to about 30% in the titer of the protein of interest. In some embodiments, the method results an increase of about 10% to about 20% in the titer of the protein of interest. In some embodiments, the method results an increase of about 20% to about 30% in the titer of the protein of interest. In some embodiments, the method results an increase of about 5%, about 10%, about 15%, about 20%, about 25%, or about 30% in the titer of the protein of interest. In some embodiments, the method results an increase of about 25% to about 75% in the titer of the protein of interest. In some embodiments, the method results an increase of about 50% to about 75% in the titer of the protein of interest. In some embodiments, the method results an increase of about 75% to about 95% in the titer of the protein of interest.

In some embodiments, the increase in titer is an increase of about 10%. In some embodiments, the increase in titer is an increase of about 20%. In some embodiments, the increase in titer is an increase of about 30%. In some embodiments, the increase in titer is an increase of about 40%. In some embodiments, the increase in titer is an increase of about 50%. In some embodiments, the increase in titer is an increase of about 60%. In some embodiments, the increase in titer is an increase of about 70%. In some embodiments, the increase in titer is an increase of about 80%. In some embodiments, the increase in titer is an increase of about 90%. In some embodiments, the increase in titer is an increase of about 100%. In some embodiments, the increase in titer is an increase of more than 100%.

In some embodiments, the protein production yield or titer, which can be expressed in grams of protein product per liter of culture medium, from cells cultured according of the disclosure is at least 100 mg/L, at least 1 g/L, at least 1.2 g/L, at least 1.4 g/L, at least 1.6 g/L, at least 1.8 g/L, at least 2 g/L, at least 2.5 g/L, at least 3 g/L, at least, 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 15 g/L, or at least 20 g/L.

In some embodiments, the method results in an increase in VCD. In some embodiments, the method results an increase of about 5% to about 30% in VCD. In some embodiments, the method results an increase of about 5% to about 20% in VCD. In some embodiments, the method results an increase of about 5% to about 15% in VCD. In some embodiments, the method results an increase of about 5% to about 10% in VCD. In some embodiments, the method results an increase of about 10% to about 30% in VCD. In some embodiments, the method results an increase of about 10% to about 20% in VCD. In some embodiments, the method results an increase of about 20% to about 30% in VCD. In some embodiments, the method results an increase of about 5%, about 10%, about 15%, about 20%, about 25%, or about 30% in VCD.

In some embodiments, the method results in an increase in cell productivity. In some embodiments, the method results an increase of about 5% to about 30% in cell productivity. In some embodiments, the method results an increase of about 5% to about 20% in cell productivity. In some embodiments, the method results an increase of about 5% to about 15% in cell productivity. In some embodiments, the method results an increase of about 5% to about 10% in cell productivity. In some embodiments, the method results an increase of about 10% to about 30% in cell productivity. In some embodiments, the method results an increase of about 10% to about 20% in cell productivity. In some embodiments, the method results an increase of about 20% to about 30% in cell productivity. In some embodiments, the method results an increase of about 5%, about 10%, about 15%, about 20%, about 25%, or about 30% in cell productivity.

In some embodiments, the method results in an increase in purity (e.g., cSDS purity). In some embodiments, the method results in an increase of about 10% to 40% in purity (e.g., cSDS purity). In some embodiments, the method results in an increase of about 20% to 40% in purity (e.g., cSDS purity). In some embodiments, the method results in an increase of about 30% to 40% in purity (e.g., cSDS purity).

In some embodiments, the method results in a decrease in glycation. In some embodiments, the method results in a decrease of about 5% to about 30% in glycation. In some embodiments, the method results in a decrease of about 5% to about 20% in glycation. In some embodiments, the method results in a decrease of about 5% to about 10% in glycation. In some embodiments, the method results in a decrease of about 10% to about 20% in glycation. In some embodiments, the method results in a decrease of about 10% to about 30% in glycation. In some embodiments, the method results in a decrease of about 20% to about 30% in glycation.

In some embodiments, the method results in improved glycation and capillary isoelectric focusing (cIEF) profile. In some embodiments, the method results in increased glycan complexity. For example, as provided herein, the method result in a lower G0F/G0F-GlcNac and increase G1F/G2F than relative to high pyruvate (e.g., sodium pyruvate) conditions.

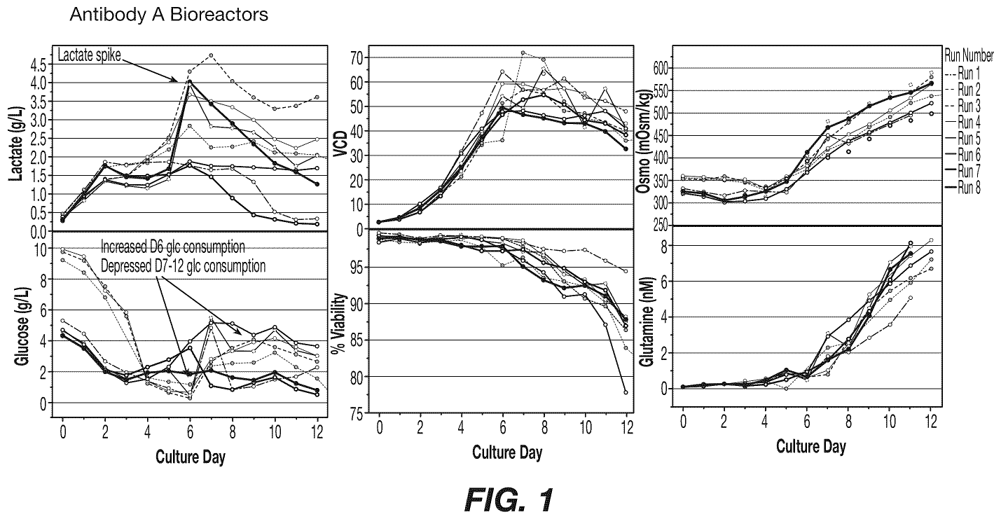

In another aspect, provided herein is a method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding one or more feeds (e.g., one or more complex feeds) during expansion phase the cells and reducing the concentration of pyruvate in one or more additional feeds (e.g., one or more additional complex feeds) before the peak cell density, wherein the pyruvate concentration in the one or more additional feeds is reduced by 65% to 100% relative to the pyruvate concentration in the one or more feeds during expansion phase of the cells. This is based, at least in part, on the observation of when the lactate spike in fed-batch culture occurred. As shown in Section 5, the lactate spike occurs during the expansion phase and before the cells reach peak viable cell density (see, e.g., FIGS. 1, 2 and 4A-4C). In a specific embodiment, provided herein is a method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding one or more feeds (e.g., one or more complex feeds) during expansion of the cells and reducing the concentration of pyruvate in one or more additional feeds (e.g., one or more additional complex feeds) before peak cell density, wherein the pyruvate concentration in the one or more additional feeds is reduced by 65% to 100% relative to the pyruvate concentration in the one or more feeds during expansion phase of the cells. In some embodiments, the peak cell density is at about 6 to about 7 days in the fed-batch culturing of the cells and the reduction in pyruvate concentration in the additional one or more feeds is at about days 3 to 5 (e.g., at about day 3, about day 4, or about 5 day) in the fed-batch culturing of the cells. In some embodiments, the concentration of one or more amino acids is increased before peak cell density.

In some embodiments, the concentration of pyruvate in the one or more additional feeds is increased by about 65% to about 95% relative to the pyruvate concentration in the one or more feeds during expansion of the cells. In some embodiments, the concentration of pyruvate (e.g., sodium pyruvate) in one or more additional feeds is reduced by about 75% to about 95% relative to the concentration of pyruvate (e.g., sodium pyruvate) found in the one or more feeds relative to the pyruvate concentration during expansion of the cells. In some embodiments, the concentration of pyruvate (e.g., sodium pyruvate) in the one or more additional feeds is reduced by about 85% to about 98% relative to the pyruvate concentration in the one or more feeds during expansion of the cells. In some embodiments, the concentration of pyruvate (e.g., sodium pyruvate) in the one or more additional feeds is reduced by about 65% or about 70% or about 65% or about 85% relative to the pyruvate concentration in the one or more feeds during expansion of the cells. In some embodiments, the concentration of pyruvate (e.g., sodium pyruvate) in the one or more additional feeds is reduced by about 75%, about 80%, about 85%, about 90%, about 95%, about 98%, about 99%, or about 100% relative to the pyruvate concentration in the one or more feeds during expansion of the cells.

In some embodiments, the increase in concentration of an amino acid in the one or more additional feeds is by 0% to 100% relative to the amino acid concentration in the one or more feeds during expansion of the cells. In some embodiments, the increase in concentration of an amino acid in the one or more additional feeds is by about 0.5% to about 10% relative to the amino acid concentration in the one or more feeds during expansion of the cells. In some embodiments, the increase in concentration of an amino acid in the one or more additional feeds is by about 0.5%, about 1%, about 2%, about 3%, about 4% or about 5% relative to the amino acid concentration in the one or more feeds during expansion of the cells. In some embodiments, the increase in concentration of an amino acid in the one or more additional feeds is by about 6%, about 7%, about 8%, about 9%, or about 10% relative to the amino acid concentration in the one or more feeds during expansion of the cells. In some embodiments, the increase in concentration of an amino acid in the one or more additional feeds is by about 0.5% to about 100% relative to the amino acid concentration in the one or more feeds during expansion of the cells. In some embodiments, the increase in concentration of an amino acid in the one or more additional feeds is by about 5% to about 100%, about 5% to about 75%, about 5% to about 50%, or about 5% to about 25% relative to the amino acid concentration in the one or more feeds during expansion of the cells. In some embodiments, the increase in concentration of an amino acid in the one or more additional feeds is by about 25% to about 100%, about 25% to about 75%, or about 25% to about 50% relative to the amino acid concentration in the one or more feeds during expansion of the cells. In some embodiments, the increase in concentration of an amino acid in the one or more additional feeds is by about 0% to about 20%, about 20% to about 40%, about 40% to about 60%, or about 20% to about 60% relative to the amino acid concentration in the one or more feeds during expansion of the cells. In some embodiments, the increase in concentration of an amino acid in the one or more additional feeds is by about 0% to about 40% about 1% to about 40%, about 60% to about 80%, or about 80% to about 100% relative to the amino acid concentration in the one or more feeds during expansion of the cells. In some embodiments, the increase in concentration of an amino acid in the one or more additional feeds is by at least 0.05%, at least 1% at least 5%, at least 10%, at least 20%, at least 20%, at least 25%, at least 30%, at least 35% or at least 40% relative to the amino acid concentration in the one or more feeds during expansion of the cells. In some embodiments, the increase in concentration of an amino acid in the one or more additional feeds is by at least 45%, at least 50% at least 55%, at least 60%, at least 65%, at least 70%, at least 75%, or at least 80% relative to the amino acid concentration in the one or more feeds during expansion of the cells. In some embodiments, the increase in concentration of an amino acid in the one or more additional feeds is by at least 85%, at least 90% at least 95%, at least 98%, at least 99%, or 100% relative to the amino acid concentration in the one or more feeds during expansion of the cells.

In some embodiments, the cells are mammalian cells. In specific embodiments, the cells are mammalian cell lines. Non-limiting examples of mammalian host cell lines suitable for use in the present disclosure include the Chinese hamster ovary (CHO), mouse myeloma derived NSO and Sp2/0 cells, human embryonic kidney cells (HEK293), and human embryonic retinoblast-derived PER.C6 cells. In some embodiments, the mammalian cell line is a CHO cell line. See Section 4.1.2 for additional information and embodiments regarding cells.

In specific embodiments, the media and feeds (e.g., complex feeds) used in the fed-batch process is appropriate for the cells being used. In specific embodiments, the media and feeds (e.g., complex feeds) used in the fed-batch process allows the cells being used to grow and produce the protein(s) of interest. See Section 4.1.4 for additional information and embodiments regarding media and feeds.

The bioreactor used for the fed-batch has a suitable volume that allows for the cultivation and propagation of biological cells capable of producing the protein(s) of interest. For example, the volume of the bioreactor can be about 10 milliliters (mL) to about 25,000 L. In another example, the volume of the bioreactor can be about 0.5 liters (L) to about 25,000 L. In some embodiments, the volume of the bioreactor can be about 10 mL. In some embodiments, the volume of the bioreactor can be about 10 mL about 100 mL. In some embodiments, the volume of the bioreactor can be about 100 mL to about 200 mL. In some embodiments, the volume of the bioreactor can be about 100 mL to about 300 mL. In some embodiments, the volume of the bioreactor can be about 100 mL to about 500 mL. In some embodiments, the volume of the bioreactor can be about 500 mL to about 750 mL. In some embodiments, the volume of the bioreactor can be about 500 mL to about 1000 mL. In some embodiments, the volume of the bioreactor can be about 500 mL to about 2 L. In some embodiments, the volume of the bioreactor can be about 1 L to about 5 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 3 L. In some embodiments, the volume of the bioreactor can be less than or equal to about 5 L. In some embodiments, the volume of the bioreactor can be less than or equal to about 10 L. In some embodiments, the volume of the bioreactor can be less than or equal to about 25 L. In some embodiments, the volume of the bioreactor can be less than or equal to about 50 L. In some embodiments, the volume of the bioreactor can be less than or equal to about 100 L. In some embodiments, the volume of the bioreactor can be less than or equal to about 250 L. In some embodiments, the volume of the bioreactor can equal to or above 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be less than or equal to about 5,000 L. In some embodiments, the volume of the bioreactor can be less than or equal to about 10,000 L. In some embodiments, the volume of the bioreactor can be less than or equal to about 15,000 L. In some embodiments, the volume of the bioreactor can be less than or equal to about 25,000 L. See Section 4.1.3 for additional information and embodiments regarding bioreactors.

In some embodiments, the method results an increase of about 5% to about 30% in the titer of the protein of interest. In some embodiments, the method results an increase of about 5% to about 20% in the titer of the protein of interest. In some embodiments, the method results an increase of about 5% to about 15% in the titer of the protein of interest. In some embodiments, the method results an increase of about 5% to about 10% in the titer of the protein of interest. In some embodiments, the method results an increase of about 10% to about 30% in the titer of the protein of interest. In some embodiments, the method results an increase of about 10% to about 20% in the titer of the protein of interest. In some embodiments, the method results an increase of about 20% to about 30% in the titer of the protein of interest. In some embodiments, the method results an increase of about 5%, about 10%, about 15%, about 20%, about 25%, or about 30% in the titer of the protein of interest. In some embodiments, the method results an increase of about 25% to about 75% in the titer of the protein of interest. In some embodiments, the method results an increase of about 50% to about 75% in the titer of the protein of interest. In some embodiments, the method results an increase of about 75% to about 95% in the titer of the protein of interest.

In some embodiments, the increase in titer is an increase of about 10%. In some embodiments, the increase in titer is an increase of about 20%. In some embodiments, the increase in titer is an increase of about 30%. In some embodiments, the increase in titer is an increase of about 40%. In some embodiments, the increase in titer is an increase of about 50%. In some embodiments, the increase in titer is an increase of about 60%. In some embodiments, the increase in titer is an increase of about 70%. In some embodiments, the increase in titer is an increase of about 80%. In some embodiments, the increase in titer is an increase of about 90%. In some embodiments, the increase in titer is an increase of about 100%. In some embodiments, the increase in titer is an increase of more than 100%.

In some embodiments, the protein production yield or titer, which can be expressed in grams of protein product per liter of culture medium, from cells cultured according of the disclosure is at least 100 mg/L, at least 1 g/L, at least 1.2 g/L, at least 1.4 g/L, at least 1.6 g/L, at least 1.8 g/L, at least 2 g/L, at least 2.5 g/L, at least 3 g/L, at least, 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 15 g/L, or at least 20 g/L.

In some embodiments, the method results in an increase in VCD. In some embodiments, the method results an increase of about 5% to about 30% in VCD. In some embodiments, the method results an increase of about 5% to about 20% in VCD. In some embodiments, the method results an increase of about 5% to about 15% in VCD. In some embodiments, the method results an increase of about 5% to about 10% in VCD. In some embodiments, the method results an increase of about 10% to about 30% in VCD. In some embodiments, the method results an increase of about 10% to about 20% in VCD. In some embodiments, the method results an increase of about 20% to about 30% in VCD. In some embodiments, the method results an increase of about 5%, about 10%, about 15%, about 20%, about 25%, or about 30% in VCD.

In some embodiments, the method results in an increase in cell productivity. In some embodiments, the method results an increase of about 5% to about 30% in cell productivity. In some embodiments, the method results an increase of about 5% to about 20% in cell productivity. In some embodiments, the method results an increase of about 5% to about 15% in cell productivity. In some embodiments, the method results an increase of about 5% to about 10% in cell productivity. In some embodiments, the method results an increase of about 10% to about 30% in cell productivity. In some embodiments, the method results an increase of about 10% to about 20% in cell productivity. In some embodiments, the method results an increase of about 20% to about 30% in cell productivity. In some embodiments, the method results an increase of about 5%, about 10%, about 15%, about 20%, about 25%, or about 30% in cell productivity.

In some embodiments, the method results in an increase in purity (e.g., cSDS purity). In some embodiments, the method results in an increase of about 10% to 40% in purity (e.g., cSDS purity). In some embodiments, the method results in an increase of about 20% to 40% in purity (e.g., cSDS purity). In some embodiments, the method results in an increase of about 30% to 40% in purity (e.g., cSDS purity).

In some embodiments, the method results in a decrease in glycation. In some embodiments, the method results in a decrease of about 5% to about 30% in glycation. In some embodiments, the method results in a decrease of about 5% to about 20% in glycation. In some embodiments, the method results in a decrease of about 5% to about 10% in glycation. In some embodiments, the method results in a decrease of about 10% to about 20% in glycation. In some embodiments, the method results in a decrease of about 10% to about 30% in glycation. In some embodiments, the method results in a decrease of about 20% to about 30% in glycation.

In some embodiments, the method results in improved glycation and capillary isoelectric focusing (cIEF) profile. In some embodiments, the method results in increased glycan complexity. For example, as provided herein, the method result in a lower GOF/GOF-GlcNac and increase G1F/G2F than relative to high pyruvate (e.g., sodium pyruvate) conditions.

In another aspect, provided herein is a protein of interest produced by a method described herein (e.g., in Section 5). In a specific aspect, provided herein is a fed-batch process described in Section 5 with a reduced lactate spike. The fed-batch processes described herein may be used to recombinantly produce a protein of interest.

### 4.1.1 Protein of Interest

The protein of interest can be any protein that one of skill in the art desires to produce using a fed-batch process. In a specific embodiment, the protein of interest may be any protein capable of being recombinantly expressed (e.g., any recombinant protein). In some embodiments, the protein of interest is antigen binding protein. In some embodiments, the protein of interest is an antibody or antigen-binding fragment thereof. In some embodiments, the protein of interest is an antibody. The antibody may be monospecific, bispecific, or multi-specific (e.g., trispecific). The antibody may be monoparatopic, biparatopic, or multiparatopic. The antibody may be an antibody fusion protein. The antibody or antigen binding fragment thereof may bind to any antigen (e.g., an infectious disease antigen, a cancer antigen, or an antigen associated with another disease or disorder). In some embodiments, the protein of interest is a cytokine. In some embodiments, the cytokine is IL-1β, IL-2, IL-5, IL-6, IL-7, IL-9, IL-12, IL-15, IL-17, IL23, TNF-alpha, or interferon (e.g., IFN-alpha, IFN-beta, or IFN-gamma). In some embodiments, the protein of interest is a fusion protein. In some embodiments, the protein of interest is an antigen. In some embodiments, the protein of interest is an enzyme (e.g., human N-acetylgalactosamine-6-sulfatase (rhGALNS)), or glucocerebrosidase). The enzyme may be one used in the food industry, such as, e.g., an enzyme used in dairy, bakery, brewing, or wine making, as emulsifier, or in sweetener production. The enzyme may be used in the pharmaceutical industry, such as, e.g., a recombinant enzyme used as a biocatalysts for the preparation of chiral drug intermediates, or as recombinant enzyme drug (e.g., a recombinant enzyme used in enzyme replacement). In some embodiments, the protein of interest is an enzyme used for enzyme replacement, such as a lysosomal storage disease enzyme. In some embodiments, the protein of interest is Agalsidase beta, Agalsidase alfa, Imiglucerase, Taliglucerase alfa, Velaglucerase alfa, Alglucerase, Sebelipase alpha, Laronidase, Idursulfase, Elosulfase alpha, Galsulfase, or Alglucosidase alpha. The protein of interest may be also tissue plasminogen activator (tpa), an interferon (e.g., IFN-alpha, IFN-beta, or IFN-gamma), a coagulation factor, erythropoietin, or an anti-nerve agent (e.g., recombinant human butyrylcholinesterase to protect against organophosphate poisoning). In specific embodiments, the protein of interest is a secreted protein. In some embodiments, the protein of interest is a membrane-bound protein. In some embodiments, the protein of interest is an intracellularly expressed protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

As used herein, the term “antibody,” is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal antibodies, monovalent antibodies, multivalent antibodies, multispecific antibodies (e.g., bispecific, trispecific antibodies so long as they exhibit the desired biological activity) including—but not limited to—fusion molecules (e.g., IgG-scFv, scFv-Fc-scFv, VHH-Fc, Fc-VHH, Fc-scFv, HC-VHH, scFv-Fc-VHH, HC-scFv), single chain antibodies, and fragments thereof (e.g., domain antibodies). An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997).

Antibodies also include, but are not limited to, synthetic antibodies, nanobodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F (ab) 2 fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more complementarity-determining regions (CDRs) of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In some embodiments, the antibodies may have modifications, wherein the modifications comprise cross-linkers, glycosylation, conjugated drugs, or thio-engineered thiol-linkages.

As used herein, the term “antigen” has its ordinary meaning in the art. An “antigen” includes a structure to which an antigen binding protein (e.g., an antibody) can bind. An antigen may be a polypeptide, carbohydrate, nucleic acid, lipid, hapten, or other naturally occurring or synthetic compound. In some embodiments, the antigen is a polypeptide. In certain embodiments, an antigen is associated with a cell, for example, is present on or in a cell. In some embodiments, an antigen is associated with a cancer cell, for example, is present on or in a cancer cell. In certain embodiments, an antigen is associated with a pathogen, such as a virus, bacteria, fungus, or a parasite.

As used herein, the term “antigen binding protein” refers to a protein that binds to an antigen. An antibody is an example of an antigen binding protein. Antigen binding proteins include, but are not limited to, e.g., a single chain antibody, a nanobody, a multidomain antibody, scFv, a Fab, and a diabody.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site, such as an antigen-binding site on an antibody, and a single epitope of a target molecule, such as an antigen, is the affinity of the antibody for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

An antigen binding protein that binds to or specifically binds to an antigen can be identified, for example, by immunoassays (e.g., ELISA, radioimmunoassays, and electrochemiluminescence immunoassays), Octet®, surface plasmon resonance (e.g., BiacoreBIACore®), or other techniques known to those of skill in the art. In some embodiments, an binding protein specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antigen binding protein to a “non-target” protein is less than about 10% of the binding of the antigen binding protein to its particular target antigen, for example, as determined by fluorescence activated cell sorting (FACS) analysis or RIA. An antigen binding protein that binds to an antigen includes one that is capable of binding the antigen with sufficient affinity such that the antigen binding protein is useful, for example, as a therapeutic and/or diagnostic agent in targeting the antigen. In certain embodiments, an antigen binding protein that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antigen binding protein binds to an epitope of an antigen that is conserved among the antigen from different species.

In certain embodiments, an antigen binding protein may comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, an antigen binding protein may comprise portions of “humanized” forms of nonhuman (e.g., camelid, murine, non-human primate) antibodies that include sequences from human immunoglobulins (e.g., recipient antibody) in which the native complementarity determining region (CDR) residues are replaced by residues from the corresponding CDR of a nonhuman species (e.g., donor antibody), such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, an antigen binding protein may comprise portions of a “fully human antibody” or “human antibody,” wherein the terms are used interchangeably herein and refer to an antibody that comprises a human variable region and, for example, a human constant region. The antigen binding protein may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147 (1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6 (5): 561-66 (1995); Brüggemann and Taussing, Curr. Opin. Biotechnol. 8 (4): 455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, an antigen binding protein may comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, an antigen binding protein may comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

As used herein, the terms “Fab” and “Fab region” have their ordinary meaning in the art. Typically, the term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

As used herein, the terms “variable region” and “variable domain” in the context of an antibody have their ordinary meaning in the art. Typically, the term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

As used herein, the term “heavy chain” in the context of an antibody has its ordinary meaning in the art. Typically, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. The sequences of heavy chains from various species are known in the art (see, e.g., IMGT®, the international ImMunoGeneTics Information System®, imgt.org).

As used herein, the term “light chain” in the context of an antibody has its ordinary meaning in the art. Typically, the term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. The sequences of light chains from various species are known in the art (see, e.g., IMGT®, the international ImMunoGeneTics Information System®, imgt.org). As used herein, the term “constant region” or “constant domain” in the context of an antibody has its ordinary meaning in the art. Typically, the term “constant region” or “constant domain” refers to a carboxy terminal portion of the light and heavy chain which is not directly involved in binding of the antibody to antigen but exhibits various effector function, such as interaction with the Fc receptor. Typically, the term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

As used herein, the term “framework” or “FR” in the context of an antibody has its ordinary meaning in the art. Typically, the term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term “Fc region” of an antibody has its ordinary meaning in the art. Typically, the term “Fc region” is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about an 80% homology with a native sequence Fc region and/or with an Fc region of a parent polypeptide, or at least about a 90% homology therewith, for example, at least about 95% homology therewith.

In certain embodiments, homology is determined by sequence similarity. Modern protein sequence databases are very comprehensive. Widely used similarity searching programs, such as BLAST (Altschul et al. (1997); units 3.3 and 3.4), PSI-BLAST (Altschul et al., 1997), SSEARCH (Smith and Waterman (1981); Pearson (1991), unit 3.10), FASTA (Pearson and Lipman (1988) unit 3.9) and the HMMER3 (Johnson et al., 2010) programs produce accurate statistical estimates, ensuring protein sequences that share significant similarity also have similar structures.

The term “specificity” in the context of an antibody or other antibody binding protein refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

Various commercially available antibodies are provided in Table 1 below, and are suitable for production in a fed-batch process described herein, either as an intact antibody, as an antibody fragment, or as a portion of a multi-specific antibody (e.g., a fusion protein). In addition, an antibody that binds to the same target as provided in Table 1 is suitable for production in a fed-batch process described herein.

TABLE 1
Exemplary Commercially available antibodies

| Antibody | Brand name | Company | Target | Indication |
|---|---|---|---|---|
| Muromonab- | Orthoclone | Centocor Ortho | CD3 | Kidney transplant |
| CD3 | OKT3 | Biotech Products LP. | | rejection |
| Abciximab | Reopro | Centocor Inc./Eli | GPIIb/IIIa | Prevention of blood clots |
| | | Lilly/Janssen Biotech Inc. | | in angioplasty |
| Rituximab | MabThera, | Biogen Inc./Roche, F. | CD20 | Non-Hodgkin lymphoma |
| | Rituxan | Hoffmann-La Roche | | |
| | | Ltd./Genentech Inc. | | |
| Palivizumab | Synagis | MedImmune/AbbVie Inc. | RSV | Prevention of respiratory |
| | | | | syncytial virus infection |
| Infliximab | Remicade | Janssen Biotech Inc. | TNFα | Crohn's disease |
| Trastuzumab | Herceptin | Roche, F. | HER2 | Breast cancer |
| | | Hoffmann-La Roche, | | |
| | | Ltd./Genentech Inc. | | |
| Alemtuzumab | Campath, | Berlex Inc./Genzyme | CD52 | Chronic myeloid leukemia |
| | Lemtrada | Corp./Millennium | | |
| | | Pharmaceuticals Inc. | | |
| Adalimumab | Humira | AbbVie Inc. | TNFα | Rheumatoid arthritis |
| Ibritumomab | Zevalin | Biogen Inc./Schering | CD20 | Non-Hodgkin lymphoma |
| tiuxetan | | AG/Spectrum | | |
| | | Pharmaceuticals Inc. | | |
| Omalizumab | Xolair | Roche, F. Hoffmann-La | IgE | Asthma |
| | | Roche, Ltd./Genentech | | |
| | | Inc./Novartis | | |
| | | Pharmaceuticals | | |
| | | Corp./Tanox Inc. | | |
| Cetuximab | Erbitux | Bristol-Myers | EGFR | Colorectal cancer |
| | | Squibb/Merck & Co. | | |
| | | Inc./Eli Lilly/ImClone | | |
| | | Systems Inc. | | |
| Bevacizumab | Avastin | Roche, F. | VEGF-A | Colorectal cancer |
| | | Hoffmann-La Roche, | | |
| | | Ltd./Genentech Inc. | | |
| Natalizumab | Tysabri | Biogen Inc./Elan | ITGA4 | Multiple sclerosis |
| | | Pharmaceuticals | | |
| | | International, Ltd. | | |
| Panitumumab | Vectibix | Amgen | EGFR | Colorectal cancer |
| Ranibizumab | Lucentis | Roche, F. Hoffmann-La | VEGF-A | Macular degeneration |
| | | Roche Ltd./Genentech | | |
| | | Inc./Novartis | | |
| | | Pharmaceuticals Corp. | | |
| Eculizumab | Soliris | Alexion | C5 | Paroxysmal nocturnal |
| | | Pharmaceuticals Inc. | | hemoglobinuria |
| Certolizumab | Cimzia | Celltech, UCB. | TNFα | Crohn's disease |
| pegol | | | | |
| Ustekinumab | Stelara | Medarex/Centocor Ortho | IL-12/23 | Psoriasis |
| | | Biotech Inc./Janssen | | |
| | | Biotech Inc. | | |
| Canakinumab | Ilaris | Novartis | IL-1β | Muckle-Wells syndrome |
| | | Pharmaceuticals Corp. | | |
| Golimumab | Simponi | Centocor Ortho | TNFα | Rheumatoid and psoriatic |
| | | Biotech Inc./Janssen | | arthritis, ankylosing |
| | | Biotech Inc. | | spondylitis |
| Ofatumumab | Arzerra | Genmab A/S/ | CD20 | Chronic lymphocytic |
| | | GlaxoSmithKline/ | | leukemia |
| | | Novartis. | | |
| Tocilizumab | RoActemra, | Chugai Pharmaceutical | IL-6R | Rheumatoid arthritis |
| | Actemra | Co., Ltd./Roche, F. | | |
| | | Hoffmann-La Roche. | | |
| | | Ltd./Genentech Inc. | | |
| Denosumab | Xgeva, | Amgen | RANKL | Bone loss |
| | Prolia | | | |
| Belimumab | Benlysta | GlaxoSmithKline/Human | BLyS | Systemic lupus |
| | | Genome Sciences Inc. | | erythematosus |
| Ipilimumab | Yervoy | Bristol-Myers | CTLA-4 | Metastatic melanoma |
| | | Squibb/Medarex | | |
| Brentuximab | Adcetris | Seattle genetics | CD30 | Hodgkin lymphoma, |
| vedotin | | Inc./Takeda | | systemic anaplastic large |
| | | Pharmaceutical Co., Ltd. | | cell lymphoma |
| Pertuzumab | Perjeta | Roche, F. | HER2 | Breast Cancer |
| | | Hoffmann-La Roche, | | |
| | | Ltd./Genentech Inc. | | |
| Trastuzumab | Kadcyla | Roche, F. Hoffmann-La | HER2 | Breast cancer |
| emtansine | | Roche Ltd./Genentech | | |
| | | Inc./ImmunoGen Inc. | | |
| Raxibacumab | Abthrax | GlaxoSmithKline/Human | B. anthrasis | Anthrax infection |
| | | Genome Sciences Inc. | P A | |
| | | (HGSI) | | |
| Obinutuzumab | Gazyva, | Biogen Inc./Roche, F. | CD20 | Chronic lymphocytic |
| | Gazyvaro | Hoffmann-La Roche, | | leukemia |
| | | Ltd./Genentech Inc. | | |
| Siltuximab | Sylvant | Centocor Inc./Janssen | IL-6 | Castleman disease |
| | | Biotech Inc./Janssen- | | |
| | | Cilag International NV | | |
| Ramucirumab | Cyramza | Eli Lilly/ImClone | VEGFR2 | Gastric cancer |
| | | Systems Inc. | | |
| Vedolizumab | Entyvio | Genentech | α4β7 | Ulcerative colitis, Crohn |
| | | Inc./Millennium | integrin | disease |
| | | Pharmaceuticals | | |
| | | Inc./Takeda | | |
| | | Pharmaceuticals | | |
| | | U.S.A. Inc. | | |
| Blinatumomab | Blincyto | Amgen | CD19, CD3 | Acute lymphoblastic |
| | | | | leukemia |
| Nivolumab | Opdivo | Bristol-Myers | PD-1 | Melanoma, non-small cell |
| | | Squibb/Ono | | lung cancer |
| | | Pharmaceutical Co., Ltd. | | |
| Pembrolizumab | Keytruda | Merck & Co. Inc. | PD-1 | Melanoma |
| Idarucizumab | Praxbind | Boehringer Ingelheim | Dabigatran | Reversal of dabigatran- |
| | | Pharmaceuticals | | induced anticoagulation |
| Necitumumab | Portrazza | Eli Lilly/ImClone | EGFR | Non-small cell lung cancer |
| | | Systems Inc. | | |
| Dinutuximab | Unituxin | United Therapeutics | GD2 | Neuroblastoma |
| | | Corporation | | |
| Secukinumab | Cosentyx | Novartis | IL-17α | Psoriasis |
| | | Pharmaceuticals Corp. | | |
| Mepolizumab | Nucala | Centocor | IL-5 | Severe eosinophilic |
| | | Inc./GlaxoSmithKline | | asthma |
| Alirocumab | Praluent | Regeneron | PCSK9 | High cholesterol |
| | | Pharmaceuticals | | |
| | | Inc./Sanofi. | | |
| Evolocumab | Repatha | Amgen/Amgen Astellas | PCSK9 | High cholesterol |
| | | BioPharma K.K. | | |
| Daratumumab | Darzalex | Genmab A/S/Janssen | CD38 | Multiple myeloma |
| | | Biotech Inc. | | |
| Elotuzumab | Empliciti | Bristol-Myers | SLAMF7 | Multiple myeloma |
| | | Squibb/AbbVie Inc. | | |
| Ixekizumab | Taltz | Eli Lilly | IL-17α | Psoriasis |
| Reslizumab | Cinqaero, | Celltech, UCB/ | IL-5 | Asthma |
| | Cinqair | Schering-Plough/Teva | | |
| | | Pharmaceutical | | |
| | | Industries, Ltd. | | |
| Olaratumab | Lartruvo | Eli Lilly/ImClone | PDGFRα | Soft tissue sarcoma |
| | | Systems Inc. | | |
| Bezlotoxumab | Zinplava | Merck & Co. Inc. | Clostridium | Prevention of Clostridium |
| | | | difficile | difficile infection |
| | | | enterotoxin B | recurrence |
| Atezolizumab | Tecentriq | Roche, F. | PD-L1 | Bladder cancer |
| | | Hoffmann-La Roche, | | |
| | | Ltd./Genentech Inc. | | |
| Obiltoxaximab | Anthim | Elusys Therapeutics Inc. | B. anthrasis | Prevention of inhalational |
| | | | P A | anthrax |
| Inotuzumab | Besponsa | Wyeth | CD22 | Acute lymphoblastic |
| ozogamicin | | Pharmaceuticals/Pfizer. | | leukemia |
| Brodalumab | Siliq, | MedImmune/Amgen/ | IL-17R | Plaque psoriasis |
| | Lumicef | Kyowa Hakko Kirin/ | | |
| | | AstraZeneca/Valeant | | |
| | | Pharmaceuticals | | |
| | | International Inc. | | |
| Guselkumab | Tremfya | MorphoSys/Janssen | IL-23 p19 | Plaque psoriasis |
| | | Biotech Inc. | | |
| Dupilumab | Dupixent | Regeneron | IL-4Rα | Atopic dermatitis |
| | | Pharmaceuticals | | |
| | | Inc./Sanofi | | |
| Sarilumab | Kevzara | Regeneron | IL-6R | Rheumatoid arthritis |
| | | Pharmaceuticals | | |
| | | Inc./Sanofi | | |
| Avelumab | Bavencio | Merck Serono | PD-L1 | Merkel cell carcinoma |
| | | International S.A./Pfizer | | |
| Ocrelizumab | Ocrevus | Biogen Inc./Roche, F. | CD20 | Multiple sclerosis |
| | | Hoffmann-La Roche, | | |
| | | Ltd./Genentech | | |
| | | Inc./SIGMA-TAU | | |
| | | Industrie Farmaceutiche | | |
| | | Riunite S.p.A. | | |
| Emicizumab | Hemlibra | Chugai Pharmaceutical | Factor IXa, | Hemophilia A |
| | | Co., Ltd./Roche, F. | X | |
| | | Hoffmann-La Roche, Ltd. | | |
| Benralizumab | Fasenra | MedImmune/Kyowa | IL-5Rα | Asthma |
| | | Hakko Kirin/AstraZeneca | | |
| Gemtuzumab | Mylotarg | Pfizer | CD33 | Acute myeloid leukemia |
| ozogamicin | | | | |
| Durvalumab | Imfinzi | MedImmune/AstraZeneca | PD-L1 | Bladder cancer |
| Burosumab | Crysvita | Kyowa Hakko | FGF23 | X-linked |
| | | Kirin/Ultragenyx | | hypophosphatemia |
| | | Pharmaceutical Inc. | | |
| Lanadelumab | Takhzyro | Dyax Corp. | Plasma | Hereditary angioedema |
| | | | kallikrein | attacks |
| Mogamulizumab | Poteligeo | Kyowa Hakko Kirin | CCR4 | Mycosis fungoides or |
| | | | | Sézary syndrome |
| Erenumab | Aimovig | Novartis | CGRPR | Migraine prevention |
| Galcanezumab | Emgality | Eli Lilly | CGRP | Migraine prevention |
| Tildrakizumab | Ilumya | Merck & Co. Inc./Sun | IL-23 p19 | Plaque psoriasis |
| | | Pharmaceutical | | |
| | | Industries, Ltd. | | |
| Cemiplimab | Libtayo | Regeneron | PD-1 | Cutaneous squamous cell |
| | | Pharmaceuticals Inc. | | carcinoma |
| Emapalumab | Gamifant | NovImmmune | IFNγ | Primary hemophagocytic |
| | | | | lymphohistiocytosis |
| Fremanezumab | Ajovy | Teva Pharmaceutical | CGRP | Migraine prevention |
| | | Industries, Ltd. | | |
| Ibalizumab | Trogarzo | Taimed Biologics Inc./ | CD4 | HIV infection |
| | | Theratechnologies Inc. | | |
| Moxetumomab | Lumoxiti | MedImmune/AstraZeneca | CD22 | Hairy cell leukemia |
| pasudodox | | | | |
| Ravulizumab | Ultomiris | Alexion | C5 | Paroxysmal nocturnal |
| | | Pharmaceuticals Inc. | | hemoglobinuria |
| Caplacizumab | Cablivi | Ablynx | von | Acquired thrombotic |
| | | | Willebrand | thrombocytopenia purpura |
| | | | factor | |
| Romosozumab | Evenity | Amgen/UCB | Sclerostin | Osteoporosis in |
| | | | | postmenopausal women at |
| | | | | increased risk of fracture |
| Risankizumab | Skyrizi | Boehringer Ingelheim | IL-23 p19 | Plaque psoriasis |
| | | Pharmaceuticals/ | | |
| | | AbbVie Inc. | | |
| Polatuzumab | Polivy | Roche, F. Hoffmann-La | CD79β | Diffuse large B-cell |
| vedotin | | Roche, Ltd. | | lymphoma |
| Brolucizumab | Beovu | Novartis | VEGF-A | Macular degeneration |
| | | Pharmaceuticals Corp. | | |
| Crizanlizumab | Adakveo | Novartis | P-selectin | Sickle cell disease |
| | | Pharmaceuticals Corp. | | |

An antibody that binds to an antigen of an organism provided in Table 2 is suitable for production in a fed-batch process described herein. In addition, an antigen of an organism provided in Table 2 is for production in a fed-batch process described herein. In some embodiments, the antigen of interest, which is produced in a fed-batch process described herein, is one identified in Table 3.

TABLE 2
Organism

Legionella Pneumophila
Salmonella
Staphylococcus
Ureaplasma urealyticum
Aeromonas
Arthrobacter Globiformis
Bacillus (e.g., B. thermophilus,
B. halodurans. B. alcalophilus).
Bordetella Pertussis
Borrelia (e.g., B. burgdorferi sensu
stricto, B. garinii, B. bavariensis,
B. afzelii and B. spielmanii)
Brucella Abortus
Campylobacter Jejuni
Candida Albicans
Chlamydia Trachomatis
Chlamydophila Pneumoniae
Clostridium tetani
Corynebacterium Diphtheriae
Escherichia coli
Haemophilus influenzae
Helicobacter pylori
Leptospira biflexa
Listeria monocytogenes
Mycobacterium Tuberculosis
Mycoplasma Pneumoniae
Streptococcus Pneumoniae
Streptomyces Avidinii
Treponema Pallidum
Yersinia Enterocolitica
Adenovirus
ASFV
Chikungunya Virus
Coronavirus
Coxsackie Virus
Crimean-Congo Hemorrhagic Fever Virus
Cytomegalovirus
Dengue Virus
Ebola Virus
Echovirus
Enterovirus
Epstein-Barr Virus
Hepatitis A virus (HAV)
Hepatitis B virus (HBV)
Human cytomegalovirus (HCMV)
Hepatitis C virus (HCV)
Hepatitis Delta virus (HDV)
Hepatitis E virus (HEV)
Human immunodeficiency virus (HIV)
(e.g., type 1 or 2)
Human papillomavirus (HPV)
Herpes simplex virus (HSV)
Human T-lymphotropic virus (HTLV)
(e.g., type 1 or 2)
Influenza A Virus (e.g., group 1 or 2)
Influenza B Virus
Japanese Encephalitis
Leukemia Virus
Marburg Virus
Measles Virus
Metapneumovirus
Molluscum Contagiosum
Mumps Virus
Nipah Virus
Norovirus
Orf Virus
Parainfluenza Virus
Parvovirus
Poliovirus
Rabies Virus
Respiratory Syncytial Virus
Rhinovirus
Rift Valley Fever Virus
Poliovirus
Rotavirus
Rubella Virus
Severe acute respiratory syndrome
coronavirus 1 (SARS-CoV-1)
Severe acute respiratory syndrome
coronavirus 2 (SARS-CoV-2)
Simian (Macaque) Immunodeficiency
Virus
TBEV
Tobacco Etch Virus
Varicella Zoster Virus
Variola
Venezuelan Equine Encephalitis Virus
West Nile Virus
Yellow Fever Virus
Zika Virus
Aspergillus
Saccharomyces (e.g., Saccharomyces
cerevisiae)
Leishmania
Plasmodium Falciparum
Plasmodium Vivax
Plasmodium
Toxoplasma Gondii
Caenorhabditis Elegans
Echinococcus Granulosus
Malaria
Schistosoma Japonicum
Trichomonas Vaginalis
Trypanosoma cruzi (T. cruzi)

TABLE 3

| Organism | Exemplary Antigens |
|---|---|
| Salmonella | O or R antigen |
| Staphylococcus | poly-N-succinyl β-1-6 |
| | glucosamine (PNSG), |
| | or Protein A |
| Escherichia coli | LPS (O antigens) an |
| | PS (K antigens) |
| Streptococcus Pneumoniae | M and T antigens |
| Hepatitis A virus (HAV) | Surface glycoprotein |
| Hepatitis B virus (HBV) | Hepatitis surface |
| | antigen |
| Human cytomegalovirus (HCMV) | gB or gH |
| Hepatitis C virus (HCV) | S protein or E2 |
| | protein |
| Human immunodeficiency virus | Gp120 or Nef |
| (HIV) (e.g., type 1 or 2) | |
| Human papillomavirus (HPV) | E6 or E7 |
| Herpes simplex virus (HSV) | gB, gD, and gH/gL |
| Human T-lymphotropic virus | Tax protein |
| (HTLV) (e.g., type 1 or 2) | |
| Influenza A Virus | Hemagglutinin or |
| (e.g., group 1 or 2) | neuraminidase |
| Influenza B Virus | Hemagglutinin or |
| | neuraminidase |
| Japanese Encephalitis | Envelope protein |
| Leukemia Virus | |
| Marburg Virus | Surface glycoprotein |
| Measles Virus | H, F, M, NP, or P |
| Respiratory Syncytial Virus | F or G protein |
| Severe acute respiratory | Spike protein or |
| syndrome coronavirus 1 | nucleoprotein |
| (SARS-CoV-1) | |
| Severe acute respiratory | Spike protein or |
| syndrome coronavirus 2 | nucleoprotein |
| (SARS-CoV-2) | |

A protein of interest may be used in vitro, ex vivo, or in vivo. For example, a protein of interest may have therapeutic or diagnostic uses. In another example, a protein of interest (e.g., an antigen) may be used as an immunogen. In another example, a commercial antibody described in Table 1 may be used for the indication noted in Table 1 or another approved indication. In another example, a protein of interest may be used in food industry, such as, e.g., an enzyme used in dairy, bakery, brewing, or wine making, as emulsifier, or in sweetener production. In another example, a protein of interest may be used in the pharmaceutical industry, such as, e.g., a recombinant enzyme used as a biocatalysts for the preparation of chiral drug intermediates, or as a recombinant enzyme drug (e.g., a recombinant enzyme used in enzyme replacement). In another example, a protein of interest may be used as a coagulant.

### 4.1.2 Cells

The cells used in the fed-batch process may be of any cell type, such as any animal cell types (e.g., non-human mammalian cells or human cells). In some embodiments, the cells are mammalian cells. In specific embodiments, the cells are mammalian cell lines. Non-limiting examples of mammalian cell lines suitable for use in the present disclosure include the Chinese hamster ovary (CHO), mouse myeloma derived NSO and Sp2/0 cells, human embryonic kidney cells (HEK293), and human embryonic retinal-derived PER.C6 cells. In some embodiments, the mammalian cell line is a CHO cell line, such as used in Section 5. In some embodiments, the Chinese hamster ovary (CHO) cells are—DHFR (Urlaub et al., Proc. Natl. Acad. Sci. USA 77:4216 (1980)) Other examples of mammalian cell lines include monkey kidney CV1 line transformed by SV40 (COS-7, ATCC CRL 1651); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

The cells may be engineered to express a protein of interest using techniques known to one of skill in the art. In some embodiments, the cells are recombinantly engineered to express one protein of interest. In some embodiments, the cells are recombinantly engineered to express two or more proteins of interest (e.g., 2, 3, 4, or 5 proteins of interest). Conventional techniques of molecular biology, microbiology, and immunology, which are within the skill of those working in the art, may be employed. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and II (1985); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). In some embodiments, the cells are transfected or transformed (e.g., stably transformed) with a nucleic acid molecule encoding a protein of interest. A cell transfected or transformed with a nucleic acid molecule includes progeny or potential progeny of such a cell. Progeny of such a cell may not be identical to the parent cell transfected or transformed with the nucleic acid molecule due to mutations or environmental influences that may occur in succeeding generations or integration of the nucleic acid sequence into the cell genome.

### 4.1.3 Bioreactors

A bioreactor may be any culturing vessel that is manufactured or engineered to manipulate or control environmental conditions. Such culturing vessels are well known in the art. For example, a bioreactor can be a stainless steel stirred tank bioreactor (STR), an air-lift reactor, a disposable bioreactor, or a combination thereof (e.g., a disposable bioreactor combined with the STR).

Bioreactor processes and systems have been developed to, e.g., optimize gas exchange, to supply sufficient oxygen to sustain cell growth and productivity, and to remove CO. Maintaining the efficiency of gas exchange is an important criterion for ensuring successful scale up of cell culture and protein production. Such systems are well-known to the person having skill in the art.

The bioreactor used for a fed-batch process described herein has a suitable volume that allows for the cultivation and propagation of biological cells capable of producing the protein of interest. For example, the volume of the bioreactor can be about 10 milliliters (mL) to about 25,000 L. In another example, the volume of the bioreactor can be about 0.5 liters (L) to about 25,000 L. In some embodiments, the volume of the bioreactor can be about 10 mL. In some embodiments, the volume of the bioreactor can be about 10 mL about 100 mL. In some embodiments, the volume of the bioreactor can be about 100 mL to about 200 mL. In some embodiments, the volume of the bioreactor can be about 100 mL to about 300 mL. In some embodiments, the volume of the bioreactor can be about 100 mL to about 500 mL. In some embodiments, the volume of the bioreactor can be about 500 mL to about 750 mL. In some embodiments, the volume of the bioreactor can be about 500 mL to about 1000 mL. In some embodiments, the volume of the bioreactor can be about 500 mL to about 2 L. In some embodiments, the volume of the bioreactor can be about 1 L to about 5 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 3 L. In some embodiments, the volume of the bioreactor can be less than or equal to about 5 L. In some embodiments, the volume of the bioreactor can be less than or equal to about 10 L. In some embodiments, the volume of the bioreactor can be less than or equal to about 25 L. In some embodiments, the volume of the bioreactor can be less than or equal to about 50 L. In some embodiments, the volume of the bioreactor can be less than or equal to about 100 L. In some embodiments, the volume of the bioreactor can be less than or equal to about 250 L. In some embodiments, the volume of the bioreactor can equal to or above 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be less than or equal to about 5,000 L. In some embodiments, the volume of the bioreactor can be less than or equal to about 10,000 L. In some embodiments, the volume of the bioreactor can be less than or equal to about 15,000 L. In some embodiments, the volume of the bioreactor can be less than or equal to about 25,000 L.

### 4.1.4 Media

In specific embodiments, the media used in a fed-batch process described herein is appropriate for the cells being used. In specific embodiments, the media used in the fed-batch process allows the cells being used to grow and produce the protein of interest. In specific embodiments, the feeds (e.g., complex feeds) of a fed-batch process comprise media that allows the cells being used to grow and/or produce the protein of interest. In a specific embodiment, one or more feeds are complex feeds. For example, commercially available media such as Ham's F10 (Sigma), Minimal Essential Medium ((MEM), (Sigma), RPMI-1640 (Sigma), and Dulbecco's Modified Eagle's Medium ((DMEM), Sigma) may be used in the fed-batch process. The media may be supplemented as necessary with hormones and/or other growth factors (such as, e.g., insulin, transferrin, or epidermal growth factor), salts (such as sodium chloride, calcium, magnesium, and phosphate), buffers (such as, e.g., HEPES or NaHCO₃), nucleotides (such as, e.g., adenosine and thymidine), antibiotics (such as GENTAMYCIN™ drug), trace elements (defined as inorganic compounds usually present at final concentrations in the micromolar range), and glucose or an equivalent energy source (e.g., another carbohydrate). The media may be serum-free media. Buffers are generally known in the art. The disclosure is not restricted to any particular buffer or buffers, and any one of ordinary skill in the art can select an appropriate buffer or buffer system for use with a particular cell line producing a particular protein. The culture conditions, such as temperature, pH, and the like, are those previously used with the cells selected for expression, and will be apparent to the ordinarily skilled artisan. Typically, mammalian cells are grown in culture at near neutral pHs, such as from about pH 6.5 to about pH 7.5. Typically, the temperature of the culture is controlled (e.g., the temperature is typically physiological 37° C. but can be as low as 30° C.). Typically, the oxygen concentration of the culture is 20-100% air saturated. In specific embodiments, the culture is free of contaminants.

In specific embodiments, chemically-defined liquid culture medium (i.e., a liquid culture medium in which all of the chemical components are known), an animal-derived component free liquid culture medium (i.e., a liquid culture medium that does not contain any components (e.g., proteins or serum) derived from a mammal), a serum-free liquid culture medium (i.e., a liquid culture medium that does not contain the serum of a mammal), or a serum-containing liquid culture medium used in the fed-batch process. In some examples, one or more (e.g., one, two, or three) of the liquid culture medium, the first feed medium (e.g., complex feed), and the second feed medium (e.g., complex feed) are a chemically-defined, animal component-free liquid culture medium. In some embodiments, each of the feeds comprises chemically-defined, animal component-free liquid culture medium. In some examples, each of the liquid culture medium, the first feed medium (e.g., complex feed), and the second feed medium (e.g., complex feed) are different. In some embodiments, each of the feed mediums (e.g., complex feeds) are the same. In some embodiments, the feed medium (e.g., complex feed) differs between feeds. In some embodiments, some of the feed mediums (e.g., complex feeds) differ from others.

Examples of liquid culture media that may be used in the presently described fed-batch methods include, e.g., CD CHO (ThermoFisher Scientific), CD-C4 (Ecplaza), CD OptiCHO&trade; Medium (ThermoFisher Scientific), CD OptiCHO™ MediumBalanCD™ CHO Feed 2, BalanCD™ CHO Feed 4, and HyClone™ ActiPro™.

The cells may be cultured in a bioreactor containing about 100 to about 200 mL of media, about 300 to about 1000 mL of media, about 500 mL to about 3000 mL of media, about 2000 mL to about 8000 mL of media, or about 4000 mL to about 15000 mL of media. In some embodiments, the cells may be cultured in a bioreactor containing about 10,000 to about 20,000 mL of media, about 15,000 to about 20,000 mL of media, or about 20,000 to 30,000 mL of media.

### 4.1.5 Purification of Protein of Interest

A protein of interest may be isolated or purified from fed-batch culture using techniques known to one of skill in the art. For example, a protein of interest may be purified using hydroxyapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation may also be used to purify a protein of interest depending on the protein (e.g., antibody) to be recovered. Following any preliminary purification step(s), the mixture comprising the protein (e.g., antibody) of interest and contaminants may be subjected to low pH hydrophobic interaction chromatography. In some embodiments, at least or about 5%, e.g., at least or about 10%, 15%, 20%, 25%, 30%, 40%, 45%, or 50% by weight of a protein of interest may be isolated from one or more other components present in the cell culture medium (e.g., mammalian cells or culture medium proteins) or one or more other components (e.g., DNA, RNA, or other proteins) present in a mammalian cell lysate. In some embodiments, at least or about 55%, 60%, 65%, 70%, or 75% by weight of a protein of interest may be isolated from one or more other components present in the cell culture medium (e.g., mammalian cells or culture medium proteins) or one or more other components (e.g., DNA, RNA, or other proteins) present in a mammalian cell lysate. In some embodiments, at least or about 80%, 85%, 90%, or 95% by weight of a protein of interest may be isolated from one or more other components present in the cell culture medium (e.g., mammalian cells or culture medium proteins) or one or more other components (e.g., DNA, RNA, or other proteins) present in a mammalian cell lysate.

### 4.2 Metabolic Model

In another aspect, a metabolic model of a mammalian cell line is used to model a lactate spike and identify factors capable of reducing the lactate spike. For example, in certain embodiments, the metabolic model is an in silico model. In specific embodiments, the in silico model is a Digital Twin Genome Scale Metabolic Model (see, e.g., U.S. Patent Application Publication No. 2007/0298484, the contents of which are hereby fully incorporated by reference). In some embodiments a pre-existing metabolic model of a mammalian cell line is used to model a lactate spike and identify factors capable of reducing the lactate spike. In some embodiments, a metabolic model of a mammalian cell line is created de novo and used to model a lactate spike and identify factors capable of reducing the lactate spike. In some embodiments, the model is one described in the Examples.

As provided herein, the metabolic model can be used and retrained with additional experimental data sets, such as for example one exhibiting and one not exhibiting the lactate spike. Once trained, the model is able to predict the spike, and is tested to identify one or more factors capable of reducing the lactate spike. For example, as provided herein, the model can be used to predict a metabolic factor capable of reducing the lactate spike. In some embodiments, the metabolic factor is selected from the group consisting of pyruvate, and an amino acid. In specific embodiments, the amino acid is selected from the group consisting of asparagine (Asn), glutamic acid (Glu), isoleucine (Ile), leucine (Leu), aspartic aid (Asp), valine (Val), and threonine (Thr). In some embodiments, the model is trained as described in the Examples.

In one aspect, the model provided herein is able to identify one or more metabolic factors that should be modified to lower the lactate spike. For example, the model can predict that one or more metabolic factors should be increased, and/or one or more metabolic factors should be decreased. In some embodiments, the model identifies, for example, that pyruvate concentration should be lowered while the concentrations of glutamate, isoleucine, aspartate, threonine, leucine and valine should be increased. In some embodiments, the model identifies, for example, that pyruvate concentration should be lowered while the concentrations of glutamate, leucine and valine should be increased.

In a further aspect, the modelling predictions can be applied to the bioreactors and the output (e.g., titer, rate, and/or yield) can be measured. The bioreactor can have any suitable volume that allows for the cultivation and propagation of biological cells capable of producing the desired protein. For example, the volume of the bioreactor can be about 0.5 liters (L) to about 25,000 L. In some embodiments, the volume of the bioreactor can be less than or equal to about 250 L. In some embodiments, the volume of the bioreactor can be about 0.5 liters (L) to about 250 L. In some embodiments, the volume of the bioreactor can be less than or equal to about 50 L. In some embodiments, the volume of the bioreactor can be about 1 L to about 50 L. In some embodiments, the volume of the bioreactor can be about 1 L to about 10 L. In some embodiments, the volume of the bioreactor can be about 1 L to about 5 L. In some embodiments, the volume of the bioreactor can be less than or equal to about 25 L. In some embodiments, the volume of the bioreactor can be about 1 L to about 25 L. In some embodiments, the volume of the bioreactor can be less than or equal to about 10 L. In some embodiments, the volume of the bioreactor can be less than or equal to about 5 L. In some embodiments, the volume of the bioreactor can be less than or equal to about 1 L. In some embodiments, the volume of the bioreactor can be about 1 L. In some embodiments, the volume of the bioreactor can be about 2 L. In some embodiments, the volume of the bioreactor can be about 3 L. In some embodiments, the volume of the bioreactor can be less than or equal to about 5 L. In some embodiments, the volume of the bioreactor can be less than or equal to about 10 L. In some embodiments, the volume of the bioreactor can be less than or equal to about 25 L. In some embodiments, the volume of the bioreactor can be less than or equal to about 50 L. In some embodiments, the volume of the bioreactor can be less than or equal to about 100 L. In some embodiments, the volume of the bioreactor can be less than or equal to about 250 L. In some embodiments, the volume of the bioreactor can equal to or above 1,000 L. In some embodiments, the volume of the bioreactor can be about 1,000 L to about 25,000 L. In some embodiments, the volume of the bioreactor can be about 10,000 L to about 25,000 L. In some embodiments, the volume of the bioreactor can be about 1,000 L. In some embodiments, the volume of the bioreactor can be about 2,000 L. In some embodiments, the volume of the bioreactor can be less than or equal to about 5,000 L. In some embodiments, the volume of the bioreactor can be less than or equal to about 10,000 L. In some embodiments, the volume of the bioreactor can be less than or equal to about 15,000 L. In some embodiments, the volume of the bioreactor can be less than or equal to about 25,000 L.

In some embodiments, a digital computer simulation of a fed-batch process for producing a protein of interest is implemented by one or more computing devices. Data is received that characterizes the fed-batch process. Additionally, a model of a mammalian cell line is initialized to enable a simulation of the fed-batch process to characterize a lactate spike based on the received data. The simulating identifies at least one factor contributing to the lactate spike. Data is provided that characterizes the identified at least one factor. The model can take various forms including a metabolic model that optionally includes one or more machine learning models such as neural networks. In some embodiments, the machine learning model (e.g., neural network, etc.) is trained using data extracted from representative processes exhibiting no lactate spike and representative processes exhibiting a lactate spike. The received data and/or the extracted data, in some embodiments, characterizes one or more of product composition, initial conditions, and nutrient additions of the fed-batch process. The providing of data can take various forms including displaying the identified at least one factor in a graphical user interface, storing the identified at least one factor in physical persistence, loading the identified at least one factor in memory, or transmitting the identified at least one factor over a network to a remote computing system. Further, in some embodiments, the identified at least one factor indicates that pyruvate concentration in one or more feeds (e.g., one or more complex feeds) to cells in a bioreactor in the fed-batch process need to be reduced in order to reduce the lactate spike.

In some embodiments, a protein of interest is produced as part of a fed-batch process. Data is received that characterizes the fed-batch process. Additionally, a model of a mammalian cell line is initialized to enable a simulation of the fed-batch process to characterize a lactate spike based on the received data. The simulating identifies at least one factor contributing to the lactate spike. Thereafter, one or more operational parameters of the fed-batch processes are modified based on the identified at least one factor. The modifying can include reducing pyruvate concentration in one or more feeds to cells in a bioreactor in the fed-batch process. In addition or in the alternative, the modifying can include increasing one or more amino acids in one or more feeds to cells in a bioreactor in the fed-batch process.

### 5. EXAMPLES

### 5.1 Example 1: Lactate Spike Observed in Multiple Fed-Batch Cultures

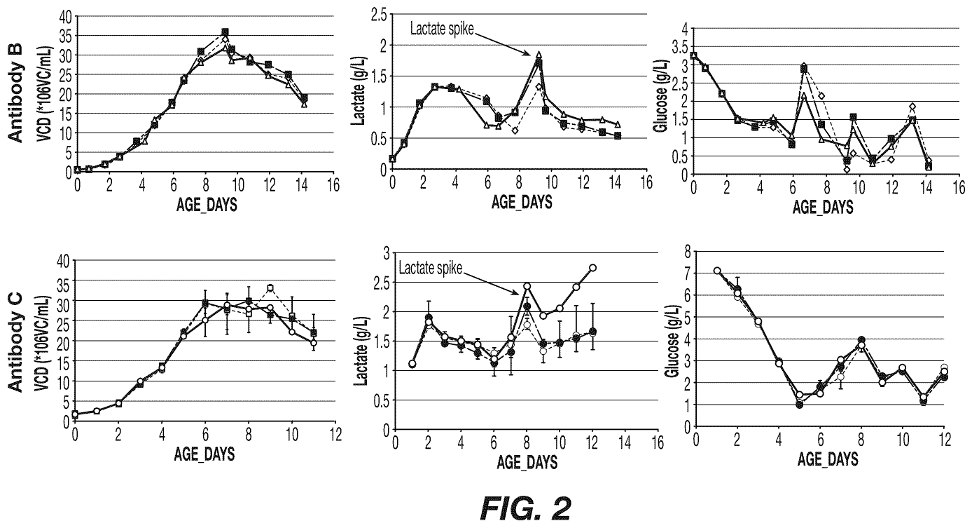

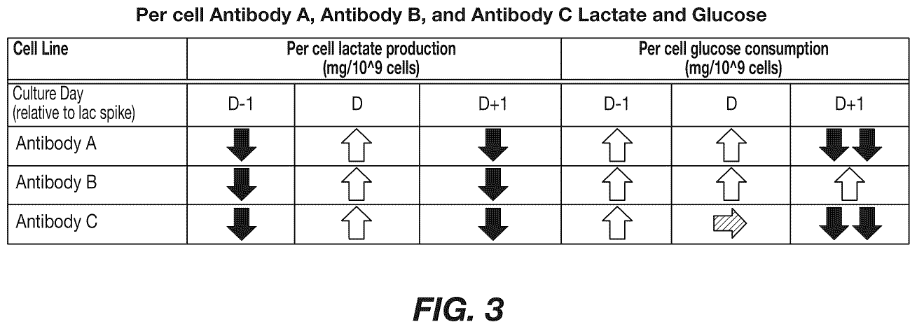

As shown in FIGS. 1 and 2, fed-batch cultures of CHO cells recombinantly expressing three different antibodies (antibody A, antibody B, and antibody C) exhibited a lactate spike protein. Antibody A cultures exhibited a lactate spike between culture days 5-6, antibody B cultures exhibited a lactate spike between culture days 8-9 and antibody C exhibited a lactate spike between culture days 7-8. As shown in FIG. 1, an increase in glucose consumption during the lactate spike followed by depressed glucose consumption, decreased oxygen was consumed, and increased culture osmolarity due to base addition was observed. However, there were no differences in VCD, viability, or glutamine concentration observed. The bioreactor conditions for the antibody A included an inoculation density of 2×10⁶ vc/ml and a bioreactor (Brx) duration of 12 days. The bioreactor conditions for the antibody B included an inoculation density of 0.5×10⁶ vc/ml and a Brx duration for 16 days. The bioreactor conditions for the antibody C included an inoculation density of 1.5×10⁶ vc/ml and a Brx duration of 12 days. The basal media for each of the three antibodies was different and the media for the complex feeds were different for two of the antibodies. As shown in FIG. 3, all cell lines showed a similar magnitude of increase in per cell lactate production and a decrease in per cell glucose consumption was observed for 2-3 cell lines post-lactate spike.

### 5.2 Example 2: Use of Genome-Based Metabolic Modeling to Identify Process Solutions to Solve Rapid Lactate Accumulation

While working with a fed-batch process for a late-phase clinical asset, a phenomenon occurred during the high productivity process where the cells producing an antibody A sporadically produced 2-3 g/L of lactate over 24 hours near peak cell density followed by consumption of the excess lactate over the remainder of the fed-batch process. Cultures sporadically exhibited abrupt lactate production between culture days 5-6. This phenomenon of rapid lactate accumulation in the middle of the fed-batch process had not been seen before and was referred to as a “lactate spike.” When this phenotype occurred, it resulted in reduced glucose metabolism, reduced product expression and poorer product quality. In particular, increased glucose consumption during lactate spike followed by depressed glucose consumption, decreased oxygen consumption, and increased culture osmolality due to base addition were observed. All cell lines tested showed a similar magnitude increase in per cell lactate production and a decrease in per cell glucose consumption was observed for 2-3 cell lines post-lactate spike. No differences in viable cell density (VCD), viability, and glutamine concentration in culture medium were observed.

The inventors tried various approaches to reduce or eliminate this lactate spike by adjusting the glucose feeding strategy and rates of a complex feed informed by spent media analysis, but these were not successful. A pre-existing metabolic model of the CHO cell line was used to model the lactate spike in order to identify targeted hypotheses that could be pursued to understand and eliminate the abrupt lactate accumulation and reduce experimental burden. In particular, a pre-existing genome-based metabolic model of the cell line was retrained with two additional experimental data sets, one exhibiting and one not exhibiting the lactate spike. Once trained, the model was able to predict the spike and was used to perform a sensitivity analysis on which media components were most likely to contribute to the lactate accumulation.

The model-based sensitivity analysis identified pyruvate along with glutamate, leucine, threonine, asparagine, aspartate, isoleucine, and valine as key feed components that were associated with the sudden lactate accumulation. The model predicted that asparagine and pyruvate concentration should be lowered while the concentrations of aspartate, threonine, glutamate, leucine, isoleucine, and valine be increased. These results were tested in 3 L bioreactors and lowering the pyruvate concentration in the complex feed was the biggest contributor to eliminating the lactate spike while concomitantly increasing titer by 20%. These two covarying results, reduced lactate and increased titer, are at odds with each other as a reduction in carbon flux through the pyruvate metabolic node would be expected to reduce flux to both lactate and the TCA cycle. Reduced flux into the TCA cycle (tricarboxylic acid cycle) would hypothetically result in reduced titer via oxidative phosphorylation but these results would indicate that reducing pyruvate improved the redox balance of the cell resulting in lower lactate and a higher productivity culture. These results were probed using the metabolic model using flux analysis to explain this counterintuitive result.

### 5.3 Example 3: Use of Digital Twins to Identify Process Solutions to Solve Rapid Lactate Accumulation

This example demonstrates that lowering pyruvate concentration in feed media eliminates sudden formation of lactate. It is proposed in this example that the lactate spike occurs due to activation of glucose uptake by high concentrations of extracellular pyruvate. The sudden lactate decline occurs due to inhibitory effects of lactate on pfk that limits glycolysis fluxes. This example demonstrates that lower pyruvate concentration in media balanced the rates of glycolytic fluxes and TCA fluxes.

Background and Objectives. During process optimization the cell culture process would sporadically produce 2-3 g/L of lactate over 24 hour period (i.e., “Lactate Spike”). Lactate spike resulted in reduced glucose metabolism, reduced product expression and lower product quality. Various approaches were tried to reduce or eliminate the lactate spike (e.g., adjusting glucose feeding and rates of complex feed addition informed by spent media analysis). These approaches were not successful at mitigating lactate accumulation, a metabolic model of the cell line was used to model the lactate spike and identify targeted hypotheses that could be pursued to eliminate the abrupt lactate accumulation.

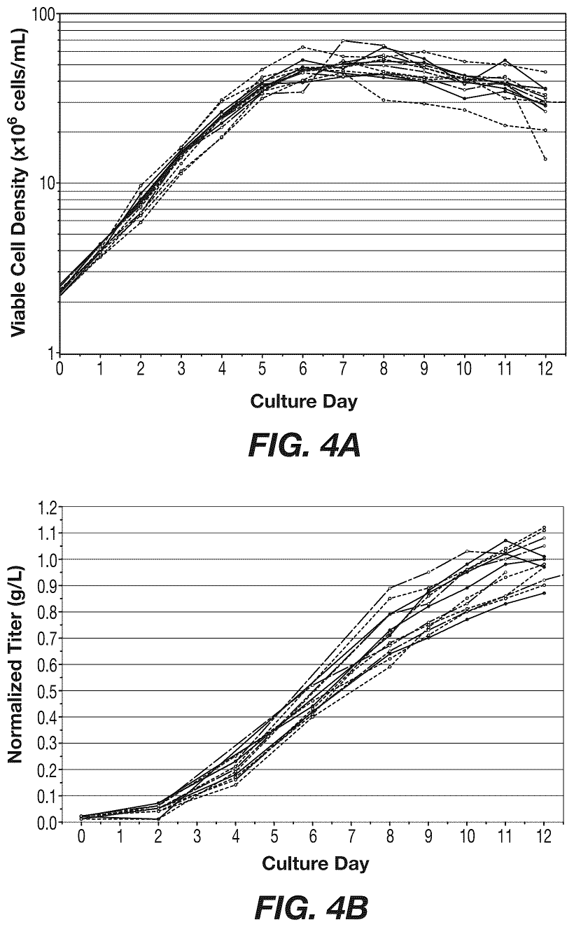

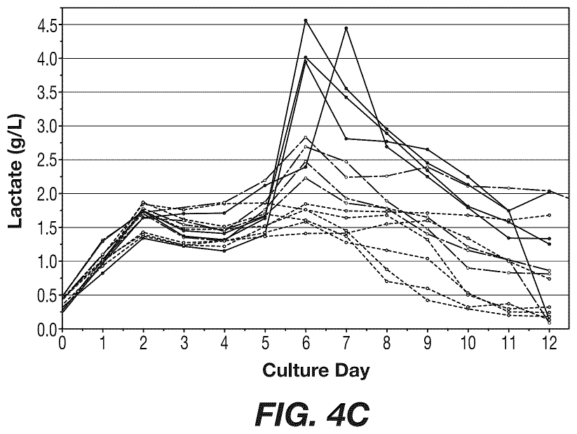

Antibody A cultures with lactate spike phenotype (FIGS. 4A-4C, (solid lines) and FIG. 1) exhibited decreased glucose consumption, decreased oxygen uptake and increased osmolality (due to base addition) following the sudden lactate accumulation. Antibody B and

Antibody C cultures also exhibited lactate spikes and changes in glucose consumption following lactate spike (FIGS. 2 and 3). Adjustments to complex and glucose feeding strategies and copper concentrations didn't prevent possibility of lactate spike occurring.

### Experimental Approach and Results

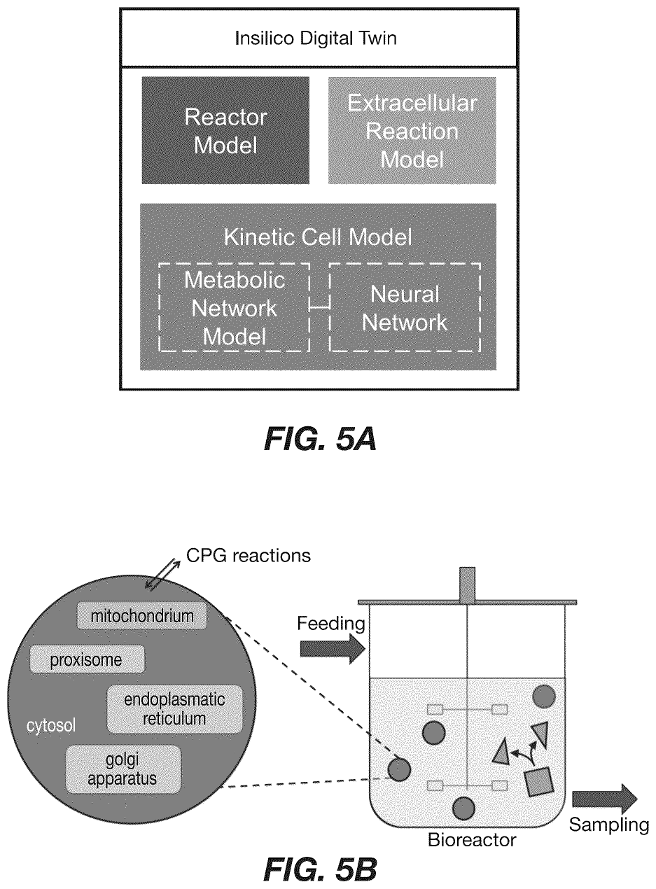

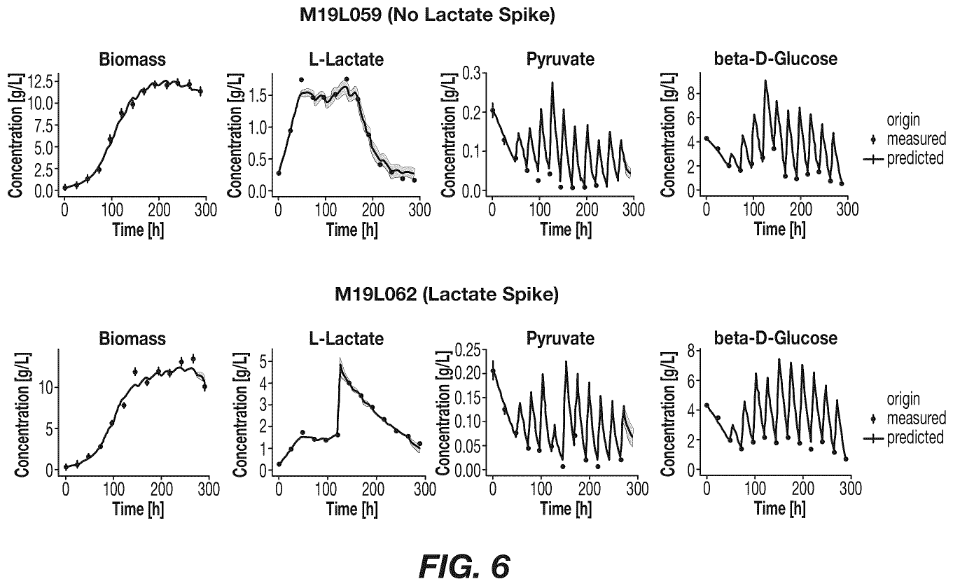

Use of in silico-Digital Twins to Identify Targeted Solutions. A Digital Twin that was developed specifically for a clone was retrained with two additional experimental data sets, one exhibiting and one not exhibiting the lactate spike. See FIGS. 5A and 5B. In particular, data (e.g., features, etc.) extracted from two representative processes displaying ‘no lactate spike’ (M19L059) or a ‘lactate spike’ (M19L062) were used to train one or more machine-learning models forming part of a metabolic model (FIG. 6). The digital twin is composed of three elements; a reactor model accounts for all component concentrations and volumes added or removed from the bioreactor, an extracellular reaction model which accounts for chemical reactions that occur extracellularly, and a kinetic cell model which describes concentration changes due to the cell metabolism. The kinetic cell model is composed of a metabolic network model (see, for example, Hefzi, H., et. al., “A Consensus Genome-Scale Reconstruction of Chinese Hamster Ovary Cell Metabolism”, Cell Systems, 3:434-443) and a recurrent neural network (RNN) model. The model inputs (i.e., the extracted features, etc.) included product composition, initial conditions (VCD, volume, pH, etc.), and nutrient additions. The digital twin is trained by first defining the number of hyperparameters in the RNN then using an 80/20 split of process data for training (80%) and testing (20%). The best hyperparameter values are then used to cross-validate the model by redistributing the process data into train-test splits. The average of five cross-validation metrics are used to determine the predictive quality of the model. Once the model was trained using similar inputs, a sensitivity analysis was performed on media components most likely to contribute to the lactate accumulation. In particular, the Digital Twin was used for sensitivity analysis on feed media components most likely to contribute to sudden lactate formation. This analysis can be used to modify one or more operational parameters of the system/process (e.g., fed-batch process, etc.). For example, the sensitivity analysis can identify at least one factor which indicates that pyruvate concentration in one or more feeds to cells in a bioreactor in the fed-batch process needs to be reduced in order to reduce the lactate spike. As another example, the sensitivity analysis can identify at least one factor indicates that the concentration of one or more amino acids in one or more feeds to cells in a bioreactor in the fed-batch process needs to be increased in order to reduce the lactate spike.

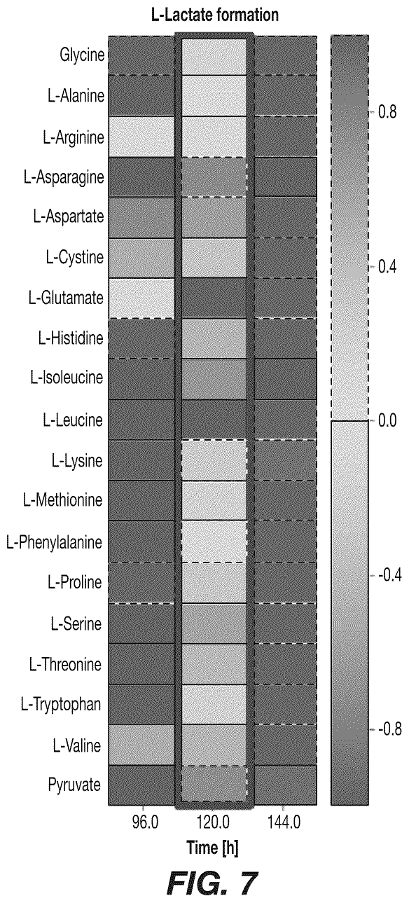

Sensitivity Analysis. Local sensitivity analysis was performed on single feed components. The heat map in FIG. 7 identifies components whose concentrations correlate to sudden lactate formation. Table 4 provides a summary of manipulations to lower lactate formation. The model-based sensitivity analysis identified pyruvate, glutamate, leucine, isoleucine, asparagine, aspartate, threonine, and valine as key media components most closely associated with sudden lactate accumulation.

TABLE 4

| For Reduced | Media Components |
|---|---|
| Lactate . . . | to Manipulate |
| Decrease | Asn, Pyruvate |
| Increase | Glu, Ile, Leu, |
| | Asp, Val, Thr |

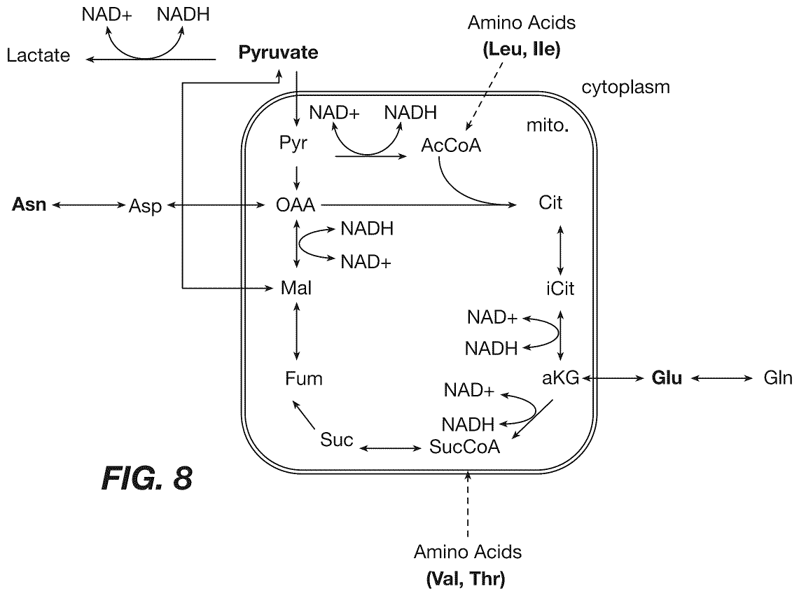

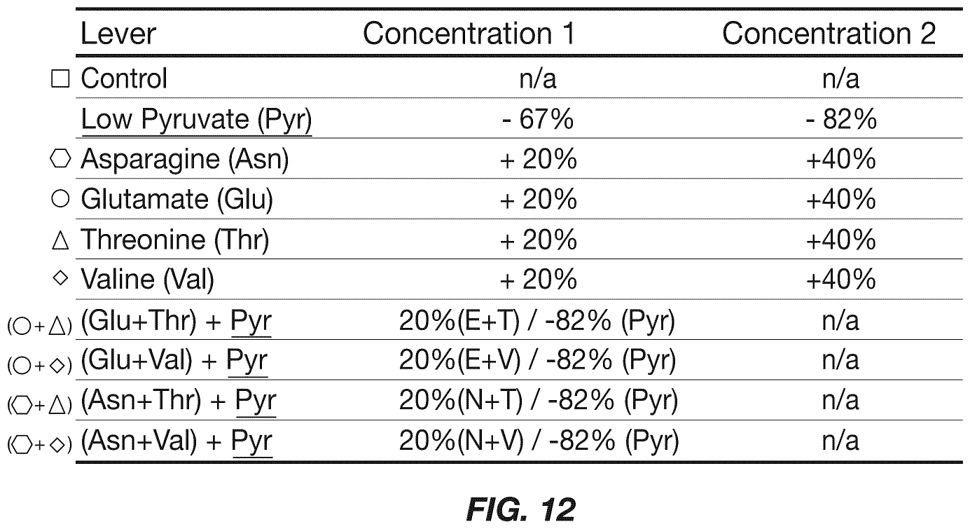

Experimental Verification of Sensitivity Analysis. Media components with strongest correlations were mapped to entry points into the TCA cycle (FIG. 8). The experiment was designed to target multiple entry points into the TCA cycle to increase likelihood of success at lowering lactate spike. See FIG. 12.

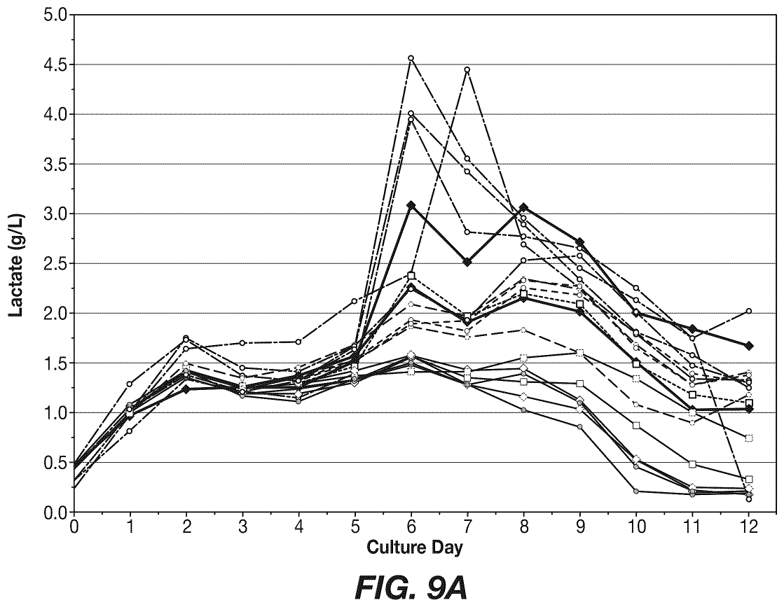

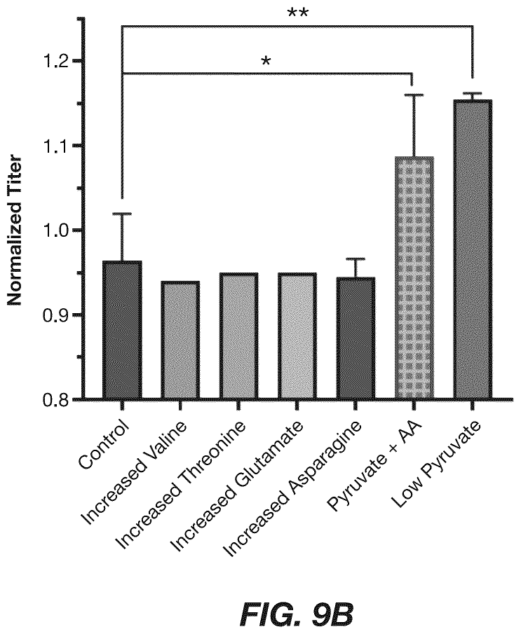

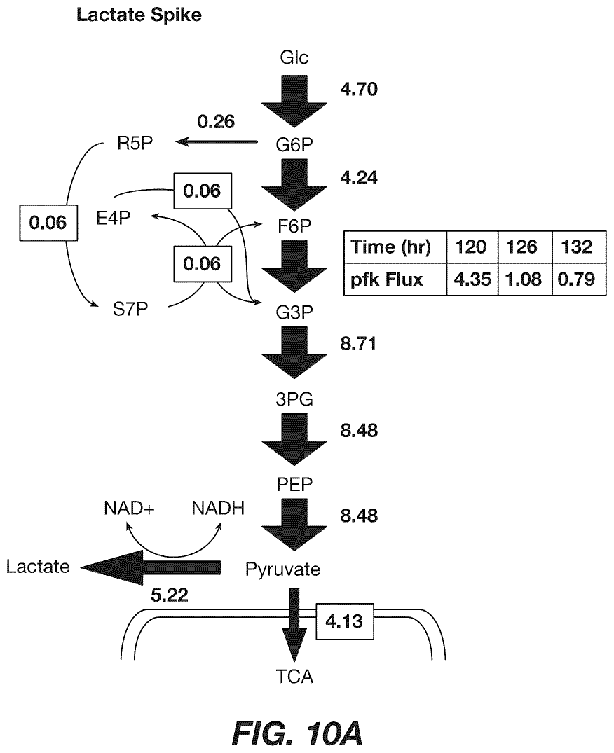

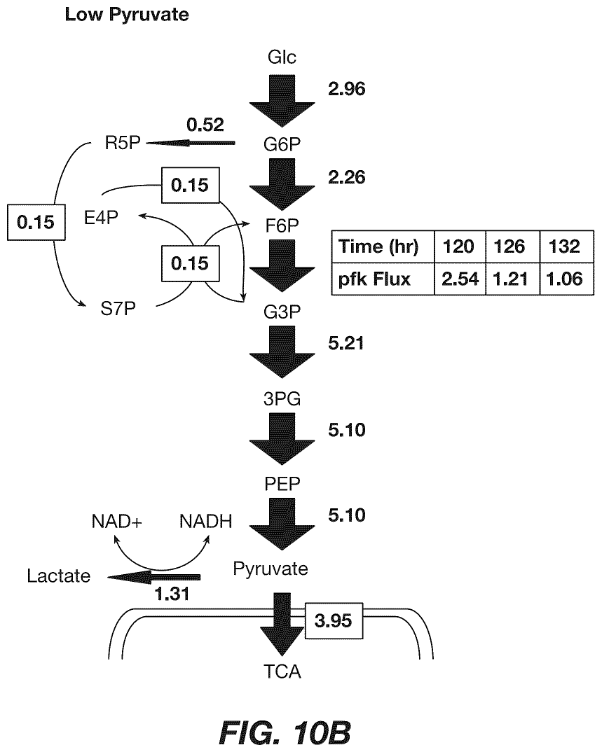

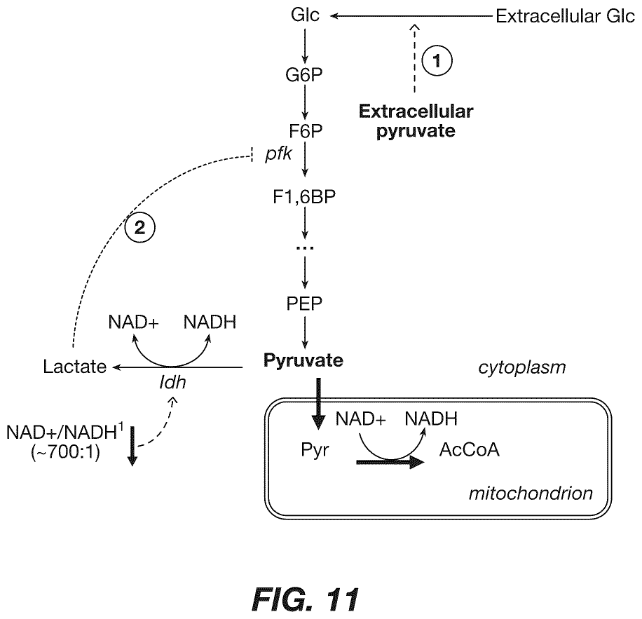

Experimental Verification Results. Results were tested in 3 L bioreactors and lowering the pyruvate concentration in the complex feed was the biggest contributor to eliminating the lactate spike. Reducing the pyruvate concentration in the feed media consistently lowered lactate and increased titer (˜20%). Reduction in pyruvate concentration resulted in increased flux into the TCA cycle leading to a 20% increase in titer. See FIGS. 9A-9B, and 10A-10B, and Table 5.

TABLE 5

| | Ratio of flux between Lactate spike |
|---|---|
| Flux at 120 h | process/Low pyruvate process |
| Pyruvate uptake | 32 |
| Glucose uptake | 1.5 |
| Lactate secretion | 4 |

### DISCUSSION

The key difference between the control and the low pyruvate media processes is that the glucose uptake rate is significantly higher (1.5×) in the control process. Surprisingly, the TCA cycle fluxes in both processes are similar. Thus lowering the pyruvate concentration in the media reduces lactate production primarily by reducing the glucose uptake rate.

Despite higher pyruvate uptake by the control process, the flux from glucose to lactate is higher in the control (60%) than the low pyruvate media process (25%). Therefore, the lactate spike in the control process is caused by higher glucose uptake.

The high glucose uptake and glycolytic fluxes consume cytosolic NAD⁺ faster than the asp-mal cycle can replenish it. The cells use the ldh reaction to produce NAD⁺ leading to a lactate spike.

Finally, the mechanism by which high concentrations of pyruvate in the media make the cells consume higher glucose is unknown. However, without being bound by theory, it is hypothesized that the effect is of a regulatory nature. The steep decrease in lactate may be due to the inhibitory effects of lactate on the enzyme pfk which reduces the flux through glycolysis. This increases the NAD+/NADH ratio and allows the cells to consume lactate back as a substrate. See FIG. 11.

Lowering pyruvate concentration in feed media eliminated sudden formation of lactate. It is proposed that the lactate spike occurs due to activation of glucose uptake by high concentrations of extracellular pyruvate. The sudden lactate decline occurs due to inhibitory effects of lactate on pfk that limits glycolysis fluxes. Lower pyruvate concentration in media balanced the rates of glycolytic fluxes and TCA fluxes. Studies to monitor intra- and extracellular redox environment to confirm role in lactate spike are planned.

### REFERENCES FOR EXAMPLE 2

1. Wilkens C A, Gerdtzen Z P. PLOS ONE. 2015; 10 (3): 1-15
2. Hartley, F., et al. Biotech and Bioeng. 2018; 115:1890-1903.
3. Mulukutla, B. C., et al. Trends in Biotechnol. 2016; 34 (8): 638-651.
4. Möller, J., et al. Eng Life Sci. 2021:100-114.

### 6. EMBODIMENTS

This invention provides the following non-limiting embodiments.

1. A method for reducing a lactate spike in a fed-batch process for producing a protein of interest, wherein the method comprises reducing pyruvate concentration in one or more feeds to cells comprising a nucleic acid encoding the protein in a bioreactor in the fed-batch process.

2. A method for increasing the titer of a protein of interest produced by cells in a fed-batch culturing process, the method comprising reducing pyruvate concentration in one or more feeds to the cells in a bioreactor in the fed-batch process.

3. The method of embodiment 1, further comprising increasing the concentration of one or more amino acids in the one or more feeds.

4. The method of embodiment 2, wherein the one or more amino acids are selected from the group consisting of glutamate, valine, leucine, threonine, aspartate, and isoleucine, or a combination thereof.

5. The method of embodiment 2, wherein the one or more amino acids are selected from the group consisting of glutamate, valine, and leucine, or a combination thereof.

6. The method of embodiment 2, wherein the one or more amino acids are glutamate, valine, or a combination thereof.

7. The method of any one of embodiments 3 to 6, wherein the amino acid concentration is increased by about 0% to about 100%.

8. The method of any one of embodiments 1 to 7, wherein the pyruvate concentration is reduced by about 65% to about 100%.

9. The method of any one of embodiments 1 to 8, wherein the cells are CHO cells.

10. The method of any one of embodiments 1 to 9, wherein the protein of interest is an antibody, a cytokine, an antigen, an enzyme, or a coagulant.

11. The method of embodiment 10, wherein the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin.

12. The method of embodiment 10, wherein the antibody binds to an antigen of a pathogen.

13. The method of embodiment 12, wherein the pathogen is a virus, a bacteria, a fungus, or a parasite.

14. The method of embodiment 10, wherein the cytokine is IL-12, IL-23, IL-1β, IL-6, IL-15, IL-2, IL-5, TNF-alpha, IL-9, or IL-17.

15. A method of producing a protein of interest, the method comprising:
(a) fed-batch culturing cells comprising a nucleic acid encoding the protein in a bioreactor under conditions sufficient for the cells to produce the protein, wherein the fed-batch culturing comprises adding a volume of one or more feeds comprising a pyruvate concentration that is 65% to 100% lower than the pyruvate concentration used in fed-batch culturing of the cells under the same conditions in which a lactate spike is observed; and
(b) purifying the protein from the cells or liquid culture medium.

16. The method of embodiment 15, wherein the concentration of one or more amino acids in the one or more feeds is increased by about 0% to about 100% relative to the fed-batch culturing of the cells under the same conditions in which a lactate spike is observed.

17. The method of embodiment 16, wherein the one or more amino acids are selected from the group consisting of glutamate, valine, leucine, threonine, aspartate, and isoleucine, or a combination thereof.

18. The method of embodiment 16, wherein the one or more amino acids are selected from the group consisting of glutamate, valine, and leucine, or a combination thereof.

19. The method of embodiment 16, wherein the one or more amino acids are glutamate, valine, or a combination thereof.

20. The method of any one of embodiments 15 to 19, wherein the cells are CHO cells.

21. The method of any one of embodiments 15 to 20, wherein the protein of interest is an antibody, a cytokine, an antigen, an enzyme, or a coagulant.

22. The method of embodiment 21, wherein the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin.

23. The method of embodiment 21, wherein the antibody binds to an antigen of a pathogen.

24. The method of embodiment 23, wherein the pathogen is a virus, a bacteria, a fungus, or a parasite.

25. The method of embodiment 21, wherein the cytokine is IL-12, IL-23, IL-1β, IL-6, IL-15, IL-2, IL-5, TNF-alpha, IL-9, or IL-17.

26. A method for providing a digital computer simulation of a fed-batch process for producing a protein of interest, the method being implemented by one or more computing devices and comprising:
receiving data characterizing the fed-batch process;
initializing a model of a mammalian cell line;
simulating, using the initialized model and the received data, the fed-batch process to characterize a lactate spike;
identifying, based on the simulating, at least one factor contributing to the lactate spike; and
providing data characterizing the identified at least one factor.

27. A method of embodiment 26, wherein the provided data characterizes one or more of product composition, initial conditions, and nutrient additions of the fed-batch process.

28. The method of embodiment 26 or 27, wherein the model is a metabolic model.

29. The method of any of embodiments 26 to 28, wherein the model comprises one or more machine learning models.

30. The method of embodiment 29, wherein the one or more machine learning models comprise: a neural network.

31. The method of embodiment 30, further comprising:
training the neural network using data extracted from representative processes exhibiting no lactate spike and representative processes exhibiting a lactate spike.

32. The method of embodiment 31, wherein the extracted data characterizes one or more of product composition, initial conditions, and nutrient additions of the fed-batch process.

33. The method of any one of embodiments 26 to 32, wherein the providing data comprises one or more:
displaying the identified at least one factor in a graphical user interface, storing the identified at least one factor in physical persistence, loading the identified at least one factor in memory, or transmitting the identified at least one factor over a network to a remote computing system.

34. The method of any one of embodiments 26 to 33, wherein the identified at least one factor indicates that pyruvate concentration in one or more feeds to cells in a bioreactor in the fed-batch process needs to be reduced in order to reduce the lactate spike.

35. The method of any one of embodiments 26 to 34, wherein the identified at least one factor indicates that the concentration of one or more amino acids in one or more feeds to cells in a bioreactor in the fed-batch process needs to be increased in order to reduce the lactate spike.

36. A method for producing a protein of interest as part of a fed-batch process comprising:
receiving data characterizing the fed-batch process;
initializing a model of a mammalian cell line;
simulating, using the initialized model and the received data, the fed-batch process to characterize a lactate spike;
identifying, based on the simulating, at least one factor contributing to the lactate spike; and
modifying one or more operational parameters of the fed-batch processes based on the identified at least one factor.

37. The method of embodiment 36, wherein the modifying comprises reducing pyruvate concentration in one or more feeds to cells in a bioreactor in the fed-batch process.

38. The method of embodiment 36 or 37, wherein the modifying comprises: increasing one or more amino acids in one or more feeds to cells in a bioreactor in the fed-batch process.

39. A method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding a volume of a first complex feed within 0 to 3 days after initiation of the fed-batch culturing of the cells, wherein the first complex feed comprises a first pyruvate concentration, and wherein the first pyruvate concentration is about 65% to about 100% lower than the pyruvate concentration used in the fed-batch culturing of the cells under the same conditions where a lactate spike is observed.

40. The method of embodiment 39, wherein the concentration of one or more amino acids is increased.

41. The method of embodiment 40, wherein the increase in the concentration of one or more amino acids is about 0% to about 100%.

42. The method of embodiment 40 or 41, wherein the one or more amino acids are selected from the group consisting of glutamate, valine, leucine, threonine, aspartate, and isoleucine, or a combination thereof.

43. The method of embodiment 40 or 41, wherein the one or more amino acids are selected from the group consisting of glutamate, valine, and leucine, or a combination thereof.

44. The method of embodiment 40 or 41, wherein the one or more amino acids are glutamate, valine, or a combination thereof.

45. The method of any one of embodiments 39 to 44, wherein the cells are CHO cells.

46. The method of any one of embodiments 39 to 45, wherein the protein of interest is an antibody, a cytokine, an antigen, an enzyme, or a coagulant.

47. The method of embodiment 46, wherein the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin.

48. The method of embodiment 46, wherein the antibody binds to an antigen of a pathogen.

49. The method of embodiment 48, wherein the pathogen is a virus, a bacteria, a fungus, or a parasite.

50. The method of embodiment 46, wherein the cytokine is IL-12, IL-23, IL-1β, IL-6, IL-15, IL-2, IL-5, TNF-alpha, IL-9, or IL-17.

51. A method for identifying the pyruvate concentration to use in one or more complex feeds in a fed-batch process, comprising:
(a) fed-batch culturing cells comprising a nucleic acid encoding a protein of interest in a first bioreactor under conditions sufficient for the cells to produce the protein, wherein the fed-batch culturing comprises adding a volume of one or more complex feeds comprising a first pyruvate concentration;
(b) fed-batch culturing the same cells in a second bioreactor under the same conditions used in the fed-batch culturing in step (a), except that the one or more complex feeds comprises a second pyruvate concentration, wherein the second pyruvate concentration is about 65% to 100% lower than the first pyruvate concentration;
(c) measuring lactate concentration in the fed-batch culturing in step (a) within about 12 to about 72 hours after each complex feed and measuring lactate concentration in the fed-batch culturing in step (b) within about 12 to about 72 hours after each complex feed; and
(d) comparing the lactate concentration measured for the fed-batch culturing in step (a) to the lactate concentration measured for the fed-batch culturing in step (b), wherein a decrease in the lactate concentration for the fed-batch culturing in step (b) relative to the lactate concentration for the fed-batch culturing in step (a) indicates that the pyruvate concentration used in the one or more complex feeds in the fed-batching culturing in step (b) are better for fed-batch culturing the cells in a bioreactor; and
(e) implementing manufacture of the protein by fed-batch culturing of the cells comprising said nucleic acid under conditions sufficient for the cells to produce the recombinant protein, wherein the fed-batch culturing comprises adding a volume of one or more complex feeds comprising the second pyruvate concentration.

52. The method of embodiment 51, wherein cells are CHO cells.

53. The method of embodiment 51 or 52, wherein the protein of interest is an antibody, a cytokine, an antigen, an enzyme, or a coagulant.

54. The method of embodiment 53, wherein the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin.

55. The method of embodiment 53, wherein the antibody binds to an antigen of a pathogen.

56. The method of embodiment 55, wherein the pathogen is a virus, a bacteria, a fungus, or a parasite.

57. The method of embodiment 53, wherein the cytokine is IL-12, IL-23, IL-1β, IL-6, IL-15, IL-2, IL-5, TNF-alpha, IL-9, or IL-17.

58. A method for reducing a lactate spike during fed-batch culturing of cells, the method comprising fed-batch culturing cells comprising a nucleic acid encoding a protein of interest in a bioreactor under conditions sufficient for the cells to produce the protein, wherein the fed-batch culturing comprises adding one or more feeds during expansion of the cells and reducing the concentration of pyruvate in one or more additional complex feeds before peak cell density, wherein the pyruvate concentration in the one or more additional complex feeds is reduced by 65% to 100% relative to the pyruvate concentration in the one or more complex feeds during expansion of the cells.

59. The method of embodiment 58, wherein the peak cell density is at about 6 to about 7 days in the fed-batch culturing of the cells and the reduction in pyruvate concentration in the one or more additional complex feeds is at about days 3 to 5 in the fed-batch culturing of the cells.

60. The method of embodiment 58 or 59, wherein the concentration of one or more amino acids in the one or more additional feeds is increased before peak cell density.

61. The method of embodiment 60, wherein the concentration of one or more amino acids in the one or more additional complex feeds is increased by 0% to 100% relative to the concentration of the one or more amino acids in the one or more complex during expansion of the cells.

62. The method of embodiment 60 or 61, wherein the one or more amino acids are selected from the group consisting of glutamate, valine, leucine, threonine, aspartate, and isoleucine, or a combination thereof.

63. The method of embodiment 60 or 61, wherein the one or more amino acids are selected from the group consisting of glutamate, valine, and leucine, or a combination thereof.

64. The method of embodiment 60 or 61, wherein the one or more amino acids are glutamate, valine, or a combination thereof.

65. The method of any one of embodiments 58 to 64, wherein the cells are CHO cells.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein including the initialized models and any resulting execution of such models may be implemented using diverse computing devices including a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet. These various embodiments may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows described herein do not necessarily require the particular order shown, or sequential order, to achieve desirable results. Other implementations can be within the scope of the following claims.

1. A method for reducing a lactate spike in a fed-batch process for producing a protein of interest, wherein the method comprises reducing pyruvate concentration in one or more feeds to cells comprising a nucleic acid encoding the protein in a bioreactor in the fed-batch process.

2. The method of claim 1, wherein the method increases the titer of the protein of interest.

3. The method of claim 1, further comprising increasing the concentration of one or more amino acids in the one or more feeds.

4. The method of claim 2, wherein the one or more amino acids are selected from the group consisting of glutamate, valine, leucine, threonine, aspartate, and isoleucine, or a combination thereof.

5. (canceled)

6. (canceled)

7. The method of claim 3, wherein the amino acid concentration is increased by about 0% to about 100%.

8. The method of claim 1, wherein the pyruvate concentration is reduced by about 65% to about 100%.

9. The method of claim 1, wherein the cells are CHO cells.

10. The method of claim 1, wherein the protein of interest is an antibody, a cytokine, an antigen, an enzyme, or a coagulant.

11. The method of claim 10, wherein
(a) the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin;
(b) wherein the antibody binds to an antigen of a pathogen;
(c) wherein the antibody binds to an antigen of a pathogen, wherein the pathogen is a virus, a bacteria, a fungus, or a parasite; or
(d) the cytokine is IL-12, IL-23, IL-1β, IL-6, IL-15, IL-2, IL-5, TNF-alpha, IL-9, or IL-17.

12. (canceled)

13. (canceled)

14. (canceled)

15. The method of claim 1, comprising:
(a) fed-batch culturing the cells in the bioreactor under conditions sufficient for the cells to produce the protein, wherein the fed-batch culturing comprises adding a volume of one or more complex feeds comprising a pyruvate concentration that is 65% to 100% lower than the pyruvate concentration used in fed-batch culturing of the cells under the same conditions in which a lactate spike is observed; and
(b) purifying the protein from the cells or liquid culture medium.

16. (canceled)

17. (canceled)

18. (canceled)

19. (canceled)

20. (canceled)

21. (canceled)

22. (canceled)

23. (canceled)

24. (canceled)

25. (canceled)

26. A method for providing a digital computer simulation of a fed-batch process for producing a protein of interest, the method being implemented by one or more computing devices and comprising:
receiving data characterizing the fed-batch process;
initializing a model of a mammalian cell line;
simulating, using the initialized model and the received data, the fed-batch process to characterize a lactate spike;
identifying, based on the simulating, at least one factor contributing to the lactate spike; and
providing data characterizing the identified at least one factor.

27. A method of claim 26, wherein the provided data characterizes one or more of product composition, initial conditions, and nutrient additions of the fed-batch process.

28. The method of claim 26, wherein the model is a metabolic model.

29. The method of claim 26, wherein the model comprises one or more machine learning models.

30. The method of claim 29, wherein the one or more machine learning models comprise: a neural network.

31. The method of claim 30, further comprising:
training the neural network using data extracted from representative processes exhibiting no lactate spike and representative processes exhibiting a lactate spike.

32. (canceled)

33. The method of claim 26, wherein the providing data comprises one or more:
displaying the identified at least one factor in a graphical user interface, storing the identified at least one factor in physical persistence, loading the identified at least one factor in memory, or transmitting the identified at least one factor over a network to a remote computing system.

34. The method of claim 26, wherein the identified at least one factor indicates (a) that pyruvate concentration in one or more feeds to cells in a bioreactor in the fed-batch process needs to be reduced in order to reduce the lactate spike or (b) that the concentration of one or more amino acids in one or more feeds to cells in a bioreactor in the fed-batch process needs to be increased in order to reduce the lactate spike.

35. (canceled)

36. A method for producing a protein of interest as part of a fed-batch process comprising: receiving data characterizing the fed-batch process;
initializing a model of a mammalian cell line;
simulating, using the initialized model and the received data, the fed-batch process to characterize a lactate spike;
identifying, based on the simulating, at least one factor contributing to the lactate spike; and
modifying one or more operational parameters of the fed-batch processes based on the identified at least one factor.

37. The method of claim 36, wherein the modifying comprises (a) reducing pyruvate concentration in one or more feeds to cells in a bioreactor in the fed-batch process; or
(b) increasing one or more amino acids in one or more feeds to cells in a bioreactor in the fed-batch process.

38. (canceled)

39. The method of claim 1, wherein the method comprises fed-batch culturing the cells in the bioreactor under conditions sufficient for the cells to produce the protein, wherein the fed-batch culturing comprises adding a volume of a first complex feed within 0 to 3 days after initiation of the fed-batch culturing of the cells, wherein the first complex feed comprises a first pyruvate concentration, and wherein the first pyruvate concentration is about 65% to about 100% lower than the pyruvate concentration used in the fed-batch culturing of the cells under the same conditions where a lactate spike is observed.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method for identifying the pyruvate concentration to use in one or more complex feeds in a fed-batch process, comprising:
(a) fed-batch culturing cells comprising a nucleic acid encoding a protein of interest in a first bioreactor under conditions sufficient for the cells to produce the protein, wherein the fed-batch culturing comprises adding a volume of one or more complex feeds comprising a first pyruvate concentration;
(b) fed-batch culturing the same cells in a second bioreactor under the same conditions used in the fed-batch culturing in step (a), except that the one or more complex feeds comprises a second pyruvate concentration, wherein the second pyruvate concentration is about 65% to 100% lower than the first pyruvate concentration;
(c) measuring lactate concentration in the fed-batch culturing in step (a) within about 12 to about 72 hours after each complex feed and measuring lactate concentration in the fed-batch culturing in step (b) within about 12 to about 72 hours after each complex feed; and
(d) comparing the lactate concentration measured for the fed-batch culturing in step (a) to the lactate concentration measured for the fed-batch culturing in step (b), wherein a decrease in the lactate concentration for the fed-batch culturing in step (b) relative to the lactate concentration for the fed-batch culturing in step (a) indicates that the pyruvate concentration used in the one or more complex feeds in the fed-batching culturing in step (b) are better for fed-batch culturing the cells in a bioreactor; and
(e) implementing manufacture of the protein by fed-batch culturing of the cells comprising said nucleic acid under conditions sufficient for the cells to produce the recombinant protein, wherein the fed-batch culturing comprises adding a volume of one or more complex feeds comprising the second pyruvate concentration.

52. The method of claim 51, wherein cells are CHO cells.

53. The method of claim 51 or 52, wherein the protein of interest is an antibody, a cytokine, an antigen, an enzyme, or a coagulant.

54. The method of claim 53, wherein
(a) the antibody binds to a glioma-associated antigen, carcinoembryonic antigen (CEA), β-human chorionic gonadotropin, alphafetoprotein (AFP), B-cell maturation antigen (BCMA), lectin-reactive AFP, thyroglobulin, RAGE-1, MN-CAIX, human telomerase reverse transcriptase, GPRC5D, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), CD70, CD20, MAGE, ELF2M, neutrophil elastase, ephrinB2, insulin growth factor (IGF)-I, IGF-II, IGF-I receptor, or mesothelin;
(b) wherein the antibody binds to an antigen of a pathogen;
(c) wherein the antibody binds to an antigen of a pathogen, wherein the pathogen is a virus, a bacteria, a fungus, or a parasite; or
(d) wherein the cytokine is IL-12, IL-23, IL-1β, IL-6, IL-15, IL-2, IL-5, TNF-alpha, IL-9, or IL-17.

55. (canceled)

56. (canceled)

57. (canceled)

58. The method of claim 1, wherein the method comprises fed-batch culturing the cells in the bioreactor under conditions sufficient for the cells to produce the protein, wherein the fed-batch culturing comprises adding one or more complex feeds during expansion of the cells and reducing the concentration of pyruvate in one or more additional complex feeds before peak cell density, wherein the pyruvate concentration in the one or more additional complex feeds is reduced by 65% to 100% relative to the pyruvate concentration in the one or more complex feeds during expansion of the cells.

59. The method of claim 58, wherein the peak cell density is at about 6 to about 7 days in the fed-batch culturing of the cells and the reduction in pyruvate concentration in the one or more additional feeds is at about days 3 to 5 in the fed-batch culturing of the cells.

60. The method of claim 58, wherein the concentration of one or more amino acids in the one or more additional feeds is increased before peak cell density.

61. (canceled)

62. (canceled)

63. (canceled)

64. (canceled)

65. (canceled)
